(19) United States
(12) Patent Application Publication
(10) Pub. No.: US 2023/0044257 A1
(43) Pub. Date: Feb. 9, 2023

(54) COMPOSITIONS AND METHODS FOR ENHANCED UPTAKE OF ACTIVE INGREDIENTS FOR ANIMAL HEALTH AND NUTRITION

(71) Applicant: AgroSpheres, Inc., Charlottesville, VA (US)

(72) Inventors: AMEER HAMZA SHAKEEL, Charlottesville, VA (US); Sepehr ZOMORODI, Charlottesville, VA (US); Sergio CARBALLO, Charlottesville, VA (US); Payam POURTAHERI, Charlottesville, VA (US); Joseph FRANK, Charlottesville, VA (US)

(21) Appl. No.: 17/788,577

(22) PCT Filed: Dec. 22, 2020
(86) PCT No.: PCT/US2020/066706

§ 371 Date: Jun. 23, 2022

Related U.S. Application Data
(60) Provisional application No. 62/952,961, filed on Dec. 23, 2019.

(51) Int. Cl. A61K 35/74; A61K 9/50; A61K 31/05

(52) U.S. Cl. CPC A61K 35/74; A61K 9/5068; A61K 31/05; A61K 9/5036

(57) ABSTRACT

The present disclosure is generally directed to a composition comprising a minicell and an active agent and to a bacterial minicell comprising an active agent. The compositions are suitable for improving animal health, increasing live stock production, or preventing animal-to-human transmission from both domestic animals and wildlife populations. Disclosed herein are methods of preparing a minicell encapsulating an active agent, delivering an active agent to a subject, and producing an animal feed and/or an animal vaccination for improving animal health and welfare.

### CROSS-REFERENCE TO RELATED APPLICATIONS

This application claims the benefit of priority to U.S. provisional application No. 62/952,961 filed on Dec. 23, 2019, which is hereby incorporated by reference in its entirety.

### FIELD

The present disclosure relates to the field of veterinary carriers and their ability to deliver active agents for enhancing animal health or the general physical condition of animals. More particularly, the disclosure relates to compositions comprising minicell systems for delivery of active agents, such as polynucleotides, polypeptides, metabolites, essential oils, and/or nutrients for improving animal health and welfare.

### BACKGROUND

Various plant-derived compounds such as secondary metabolites with beneficial effects on animal health and production, including feed and animal products. Phytobiotics can be defined as plant derived products added to feed in order to improve performance. The beneficial effects of phytobiotics in animals may arise from the activation of feed intake and the secretion of digestive enzymes, immune stimulation, antibacterial, coccidiostatic, anthelmintic, antiviral or anti-inflammatory activity, or from antioxidant properties. Phytobiotics in animal production have different applications, including sensory phytogenic additives, technological additives for improvement of feed quality and safety, as well as additives promoting animal health and welfare, acting as immunomodulators, antioxidants, digestive stimulants, and substances that can increase the performance and quality of animal products (Stevanovic et al. 2018).

Strong market pressure is being applied to align livestock farming with the concept of clean, green, and ethical (CGE). In the CGE concept, “clean” stands for reduced use of synthetic chemical substances (antibiotics, hormones, drugs), and particularly supports the idea of reducing risk of antibiotic resistance, whereas “green” focuses on reduced impact on the environment, and “ethical” refers to improvements in animal welfare (Martin et al. 2016). Phytobiotics are generally recognized as safe (GRAS). However, some bioactive metabolites of medical plants can exert toxic and even lethal effects. In addition, there is an ever-growing demand for low-priced, high quality food, improved feed hygiene, greater animal health and welfare, and reduced environmental impact.

Also, animal vaccines are important to control infectious veterinary diseases for animal health and welfare. While vaccines are the most cost-effective measure in preventing disease in livestock populations, the logistics of distributing vaccines to the populations are still a challenge. The accessibility and availability of animal vaccination needs to be address regionally and globally with an affordable price on a large scale.

Thus, there is an unmet need to develop a new delivery system as veterinary carriers to ensure active ingredients/agents to animals in a safe, non-toxic, scalable, and cost-effective manner Also, there is a great need for a novel encapsulation platform capable of controlled-release of active ingredients/agents whose bioactivity is sustained until the active compounds are directly or indirectly administered to animals for their health and welfare.

### SUMMARY OF THE DISCLOSURE

The present disclosure provides novel compositions, which can be utilized to enhance animal health and nutrition. These compositions can be developed into standalone products for application directly to an animal, or can be added into other products for enhanced animal health and welfare. Also, the present disclosure provide methods of producing and using novel compositions for animal health and welfare.

The present disclosure provides a composition comprising: a minicell and an active agent. In some embodiments, the disclosure provides composition comprising: a minicell and an active agent, wherein the active agent is encapsulated by the minicell. In some embodiments, the disclosure provides composition comprising: a minicell and an active agent, wherein the active agent is encapsulated by the minicell, and wherein the minicell and the active agent are present in a weight-to-weight ratio of about 5:1 to about 1:5 in the composition. In other embodiments, the minicell is derived from a bacterial cell. In further embodiments, the minicell is less than or equal to 1 gm in diameter. In some embodiments, the minicell confers to the active agent an improved stability, an enhanced bioavailability and an extended shelf life.

In some embodiments, the active agent is a biologically active agent. In some embodiments, the biologically active agent is selected from a nucleic acid, a peptide, a protein, an essential oil, and combinations thereof. In some embodiments, the nucleic acid is selected from the group consisting of an antisense nucleic acid, a double-stranded RNA (dsRNA), a short-hairpin RNA (shRNA), a small-interfering RNA (siRNA), a microRNA (miRNA), a ribozyme, an aptamer, and combination thereof. In some embodiments, the essential oil comprises geraniol, eugenol, genistein, carvacrol, thymol, pyrethrum or carvacrol. In some embodiments, at least about 0.1% of the active agent is encapsulated by the minicell. In some embodiments, the encapsulated active agent has an extended release when compared to a fee active agent. In some embodiments, a release of the active agent encapsulated by the biopolymer-coated minicell is further delayed when compared to the encapsulated active agent without the biopolymer coated. In some embodiments, the active agent encapsulated by the minicell is capable of being delivered to a target in a controlled release manner

The present disclosure provides a bacterial minicell comprising: an essential oil. In some embodiments, the present disclosure provides a bacterial minicell comprising: an essential oil, wherein the minicell is loaded with the essential oil in a weight-to-weight ratio of about 5:1 to about 1:5. In some embodiments, the present disclosure provides a bacterial minicell comprising: an essential oil, wherein the minicell is loaded with the essential oil in a weight-to-weight ratio of about 5:1 to about 1:5. In some embodiments, the present disclosure provides a bacterial minicell comprising: an essential oil, wherein the minicell is loaded with the essential oil in a weight-to-weight ratio of about 5:1 to about 1:5, wherein about 50% w/w to about 150% w/w of the essential oil is encapsulated by the minicell. In some embodiments, the present disclosure provides a bacterial minicell comprising: an essential oil, wherein the minicell is loaded with the essential oil in a weight-to-weight ratio of about 5:1 to about 1:5, wherein about 50% w/w to about 150% w/w of the essential oil is encapsulated by the minicell, and wherein a release percentage (%) of the encapsulated essential oil is less than about 50% in a first hour.

The present disclosure also provides a method of producing an animal feed for enhancing health of an animal, said method comprising: applying to an animal feed a composition disclosed herein. In some embodiments, the animal feed comprises at least one animal feed ingredient selected from the group consisting of a feed carbohydrate, a feed protein, a feed vitamin and mixture thereof. In some embodiments, the animal health indicator is selected from the group consisting of: gut lesion formation, gut microbiota, weight change, feed conversion ratio, and life span.

Also, the present disclosure provides a method of enhancing health of an animal, said method comprising: administering to an animal in need thereof an effective amount of a composition disclosed herein. In some embodiments, the health of the animal administered with the composition is enhanced when compared to the health of the animal not administered with the composition. In some embodiments, the composition is administered with an animal feed, which comprises at least one animal feed ingredient selected from the group consisting of a feed carbohydrate, a feed protein, a feed vitamin and mixture thereof. In some embodiments, the enhanced animal health is selected from the group consisting of: reduced or eliminated microbial infection, reduced or eliminated fungal infection, reduced or eliminated viral infection, reduced or eliminated oxidative stress, reduced or eliminated infection or death during transport, increased body weight, increased rate of weight gain, increased growth rate, reduced or eliminated birth mortality, increased or improved body score conditions, increased reproductive success, and increased gut health maturation.

Further, the present disclosure provides a method of delivering an active agent to a subject, the method comprising: applying to the subject with a composition disclosed herein. In some embodiments, the subject is an animal. In some embodiments, the active agent is selected from a nucleic acid, a peptide, a protein, an essential oil, and combinations thereof. In some embodiments, the active agent has an antimicrobial, antibacterial, antifungal or antiviral activity. In other embodiments, the active agent is a veterinary drug for animal vaccination or immunization. In further embodiments, the animal vaccination or immunization is against bacterial infections, gastric disorders, viral infections, cancer, parasite infections, non-infectious diseases, fertility and production control.

### BRIEF DESCRIPTION OF THE FIGURES

FIG. 1 illustrates a schematic representation of major benefits of incorporating active ingredients (AIs) for animal health into AgriCell (a.k.a. minicell) microencapsulation platform.

FIG. 2A-2C illustrates scanning electron microscope images of an unpurified sample of AgriCell producing E. coli. (FIG. 2A; scale bar 1 μm), a purified fraction of AgriCell showing the absence of rod-shaped parent cells (FIG. 2B; scale bar 2 μm) and magnification image showing the morphology and relatively uniform particle size of purified AgriCells (FIG. 2C; scale bar 200 nm).

FIG. 3 illustrates size distribution of an un-purified AgriCell production batch and a purified AgriCell batch. Two humps represent different populations composed by larger replicating parent cells (mean diameter about 1.0 μm) and smaller anucleate AgriCells (mean diameter about 0.5 μm). The size distribution of the purified AgriCell production shows that only small anucleate AgriCells are present (purity>99%).

FIG. 4A-4B illustrates evaluation of loading efficacy for essential oils into AgriCell (AC). FIG. 4A shows that model essential oils (EOs) (e.g. eugenol, thymol and pyrethrum, respectively) are encapsulated into AC. Bars show the correlation between original concentration of EO (200 mg/mL) and the final concentration encapsulated into AC. Line shows the percentage encapsulated EO for each formulation. FIG. 4B shows that model antimicrobial active ingredients (AIs) (e.g. eugenol and genistein, respectively) encapsulated into AgriCell (AC). Bars show the correlation between original concentration of AI (eugenol 100 mg/mL and genistein 100 mg/mL) and the final concentration encapsulated into AC. Line shows the percentage encapsulated AI for each formulation.

FIG. 5A illustrates AgriCell encapsulating eugenol (right tube), which shows improved chemical stability to changes in pH, when compared to Eugenol-encapsulating liposomal formulation (left tube). AgriCell-encapsulated eugenol showed improved stability when pH was adjusted to simulate gastric conditions (pH 1.2). FIG. 5B-5C illustrates the improved physical stability of AgriCell-encapsulated eugenol (right tube) against a Eugenol-encapsulated liposomal formulation (left tube) on day 1 (FIG. 5B) and day 30 (FIG. 5C) after storage under controlled conditions (temperature 25° C., relative humidity 30% and pH 7.2). All samples were diluted 1:10 with deionized water.

FIG. 6 illustrates evaluation of the protective effect of AgriCell on thermal degradation of essential oils at 40° C. Initial concentration of essential oil formulations was about 200 mg/mL, whereas the AgriCell concentration was about 100 mg/mL.

FIG. 7 illustrates evaluation of the protective effect of AgriCell on auto-oxidative degradation of essential oils under UV and Visible (Vis) light exposure. Initial concentration of essential oil formulations was about 200 mg/mL, whereas the AgriCell concentration was 100 mg/mL.

FIG. 8A-8B illustrates cumulative percentage release of model antimicrobial active ingredients (AIs), eugenol (FIG. 8A) and genistein (FIG. 8B) from AgriCell/minicell platform (i.e. encapsulated into AgriCell/minicell), when compared to a free AIs (eugenol and genistein, respectively) not encapsulated into AgriCell/minicell. Release media was composed by PBS, ethanol and Tween 80 emulsifier (140:59:1 v/v/v). Dialysis cassette membrane MWCO 8-10 kDa. FIG. 8A shows percentage release of (i) Eug (Eugenol 100 mg/mL) and (ii) AC-Eug (Eugenol 100 mg/mL loaded with AgriCell/Minicell platform 100 mg/mL). FIG. 8B shows percentage release of (i) Gen (Genistein 100 mg/mL) and (ii) AC-Gen (Genistein 100 mg/mL loaded with AgriCell/Minicell platform 100 mg/mL). Eugenol (100 mg/mL) and Genistein (100 mg/mL) were loaded into AgriCell/minicell platform (100 mg/mL), respectively, and encapsulated into AgriCell/minicell for investigating percentage release of eugenol and genistein in comparison to AIs in a un-coated free form (i.e. not encapsulated into AgriCell/minicell).

FIG. 9A-9C illustrates cumulative percentage release of model essential oils (EOs), eugenol (FIG. 9A), pyrethrum (FIG. 9B), and thymol (FIG. 9C) from an un-coated free form (i.e. not encapsulated into AgriCell/minicell), AgriCell/minicell platform (i.e. encapsulated into AgriCell/minicell) and AgriCell/minicell surface coated by chitosan biopolymer (MC-CHT). Release media was composed by PBS, ethanol and Tween 80 emulsifier (140:59:1 v/v/v). Dialysis cassette membrane MWCO 8-10 kDa. FIG. 9A shows percentage release of (i) Eug (Eugenol 100 mg/mL); (ii) MC-Eug (Eugenol 100 mg/mL loaded with AgriCell/Minicell platform 100 mg/mL); and (iii) MC-Eug-CHT (Eugenol 100 mg/mL loaded with AgriCell/Minicell-CHT platform (AC 100 mg/mL and CHT 20 mg/mL, weight ratio 1:1). FIG. 9B shows percentage release of (i) Pyrethrum (100 mg/mL); (ii) MC-Pyt (Pyrethrum 100 mg/mL loaded with AgriCell/Minicell platform 100 mg/mL); and (iii) MC-Pyt-CHT (Pyrethrum 100 mg/mL loaded with AgriCell/Minicell-CHT platform (AC 100 mg/mL and CHT 20 mg/mL, weight ratio 1:1). FIG. 9C shows percentage release of (i) Thymol (100 mg/mL); (ii) MC-Thym (Thymol 100 mg/mL loaded with AgriCell/Minicell platform 100 mg/mL); and (iii) MC-Thym-CHT (Thymol 100 mg/mL loaded with AgriCell/Minicell-CHT platform (AC 100 mg/mL and CHT 20 mg/mL, weight ratio 1:1).

FIG. 10A illustrates E. coli 8739 agglutination kinetics curves showing the change in transmittance (%, 450 nm) as a function of time (min) for AgriCell (300 μg/mL), genistein (300 μg/mL), and AC-genistein (Genistein 300 μg/mL loaded with AgriCell/Minicell platform 300 μg/mL, weight ratio 1:1). FIG. 10B illustrates area under the curve (AUC) obtained for the agglutination of E. coli 8739 by AgriCell (300 μg/mL), genistein (300 μg/mL), and AC-genistein (Genistein 300 μg/mL loaded with AgriCell/Minicell platform 300 μg/mL, weight ratio 1:1) at a final time point (240 min). The results are expressed as the average of three independent assays±standard deviation (n=3).

FIG. 11 illustrates zones of inhibition obtained after treatment of bacterial plates containing selected strains of Escherichia coli (left image) and Pseudomonas aeruginosa (right image) with two different doses of eugenol and AgriCell-Eugenol. 1: Sample dilution media (PBS), 2: free Eugenol (200 μg/mL), 3: AgriCell-Eugenol (Eugenol 100 μg/mL loaded with AgriCell/Minicell platform 100 μg/mL, weight ratio 1:1), 4: AgriCell-Eugenol (Eugenol 200 μg/mL loaded with AgriCell/Minicell platform 200 μg/mL, weight ratio 1:1), 5: free Eugenol (100 (+) Vancomycin (30 μg/mL), and (−) Cell culture media.

FIG. 12 illustrates bacterial inhibition effect of eugenol, (i) Eugenol: free eugenol and (ii) AC-Eugenol: eugenol encapsulated into AgriCell platform) against selected pathogenic bacterial strains (Escherichia coli and Pseudomonas aeruginosa) at two different doses (100 and 200 μg/mL), results are presented as mean±SD (n=3). The letters above the bars indicate significant differences among treatments based on Tukey's HSD test at p<0.05. Separate statistical analysis was performed for each bacterium.

FIG. 13 illustrates encapsulation efficacy for Hen egg-white protein (HEL) into AgriCell platform (100 mg/mL) as a function of HEL concentration (50 to 200 mg/mL). Data is reported as mean±SD (n=3).

FIG. 14 illustrates release profiles for free Hen egg-white protein (HEL) (100 mg/mL) and HEL encapsulated into AgriCell (100 mg/mL of HEL loaded with 100 mg/mL of AgriCell) in PBS. Results are reported as mean±SD (n=3).

FIG. 15 illustrates effects of AgriCell-HEL on interlukin-2 (IL-2) expression in 3A9 T-cell hybridomas cocultured with mouse peritoneal macrophages. Free AC (10 mg/mL), free HEL (10 mg/mL) and AgriCell-HEL (10 mg/mL)) respectively were added to the macrophage culture for 2, 4, 6, 8 and 10 hours prior to the addition of the T-cell hybridoma. Media containing the HEL and AgriCell-HEL was removed, and the T-cell hybridoma culture was added to the macrophages for 24 hours for promoting expression of IL-2.

FIG. 16 illustrates florescent microscopy experiments showing the uptake of fluorescently labeled HEL (10 mg/ml) by mouse peritoneal macrophages determined at 30 minutes after adding the protein (20× magnification). Free HEL treatment is shown on the top column and AgriCell-HEL treatment is shown one the bottom column. Phase images (brightfield) show the presence of macrophages. Fluorescent images (fluorescein) show that there is an increased uptake of the fluorescence-labeled HEL after 30 minutes when encapsulated into AgriCell platform.

FIG. 17A-17E illustrates characterization, purification, and stability of minicells encapsulated-dsRNAs (ME-dsRNAs). FIG. 17A: Scanning electron microscopy of bacterial parental and minicells. FIG. 17B: the first peak of the multisizer data represents the minicells and second peak is the parental E. coli cells. FIG. 17C: minicells from were purified from the parental cells, presenting absence of the parental cells peak. FIG. 17D: the sequence length of dsRNAs molecules encapsulated into minicells. FIG. 17E: treatment of ME- and naked-dsRNA with the RNase A. The treated and untreated ME-dsRNA were extracted prior to gel electrophoresis.

FIG. 18A-18F illustrates effects of the minicells encapsulated dsRNAs (ME-dsRNAs) on mycelial growth and silencing of respective genes in Botrytis cinerea at different time points. FIG. 18A-18B: inhibition of mycelial (fungal) growth in response to ME-dsRNAs targeting cell wall integrity related-genes chitin synthase 3a (Chs3a), and chitin synthase 3b (Chs3b) and RNAi-machinery related-genes dicer-like protein 1 (DCL1), and dicer-like protein 2 (DCL2) of B. cinerea at different concentrations. FIG. 18C-18D: mycelial growth inhibition rate in response to ME-dsRNAs (quantification of FIG. 18A-18B, respectively). FIG. 18E-18F: relative normalized expressions of the B. cinereal genes in responses to ME-dsRNAs (FIG. 18E); Chs3a and Chs3b, (FIG. 18F), DCL1 and DCL2. Data represents the mean±SEM. Bars labeled with different letters are significantly different at P<0.05 according to Duncan's multiple range test. Asterisks indicate significant differences between minicells and ME-dsRNA-treated sample at each time point according to t-test; *P<0.05, **P<0.01, ***P<0.001.

FIG. 19A-19H illustrates the lack of mycelial growth inhibitory activity of the minicells-encapsulated dsRNAs (ME-dsRNAs) targeting various genes of B. cinerea on two other fruit rot causing fungi showing the species specificity of the technology. Different concentrations of ME-dsRNAs effects on mycelial growth of Alternaria alternata (FIG. 19A-19B), and Penicillium expansum (FIG. 19E-19F) at 72 hours after treatment are presented. The mycelial growth inhibition rate of Alternaria alternata (FIG. 19C-19D; quantification of FIG. 19A-19B, respectively), and Penicillium expansum (FIG. 19G-19H; quantification of FIG. 19E-19F, respectively) are presented. Data represents the mean±SEM. Bars labeled with different letters are significantly different at P<0.05 according to Duncan's multiple range test.

### DETAILED DESCRIPTION

To improve the health and welfare of animals, new veterinary carriers for animal feeds and vaccination are required to ensure a safe, non-toxic, scalable, and cost-effective delivery of active ingredients/agents to animals.

The present disclosure relates to use of minicells as a novel delivery platform comprising active agents for the purpose of improvement of animal health and welfare. The present disclosure is directed to a composition or a formulation comprising a minicell and a active agent including a nucleic acid, a peptide, a protein, an enzyme and an essential oil. Also, disclosed are methods of preparing a minicell encapsulating an active agent, delivering an active agent via a minicell platform to an animal, and producing an animal feed and/or an animal vaccination for improving animal health and welfare.

### Definitions

While the following terms are believed to be well understood by one of ordinary skill in the art, the following definitions are set forth to facilitate explanation of the presently disclosed subject matter.

The term “a” or “an” refers to one or more of that entity, i.e. can refer to a plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein, the terms “administering” or “administration” of an active agent taught herein to a subject includes any route of introducing or delivering to a subject a compound or a composition to perform its intended function. The administering or administration can be carried out by any suitable route, including orally, intranasally, parenterally (intravenously, intramuscularly, intraperitoneally, or subcutaneously), rectally, or topically. Administering or administration includes self-administration, administration by another, or administration with other ingredients or products including an animal feed.

The term “an active agent,” (synonymous with “an active ingredient”) indicates that a composition or compound itself has a biological effect, or that it modifies, causes, promotes, enhances, blocks, reduces, limits the production or activity of, or reacts with or binds to an endogenous molecule that has a biological effect. A “biological effect” may be but is not limited to one that impacts a biological process in/onto an animal; one that impacts a biological process in and/or onto a pest, pathogen or parasite. An active agent may be used in veterinary applications. An active agent acts to cause or stimulate a desired effect upon an animal, a mammal, a human, a plant, an insect, a worm, bacteria, fungi, or virus; one that generates or causes to be generated a detectable signal; and the like. Non-limiting examples of desired effects include, for example, (i) suppressing, inhibiting, limiting, or controlling growth of or killing one or more a pest, a pathogen or a parasite that infects an animal, (ii) preventing, treating or curing a disease or condition in an animal suffering therefrom; and (iii) stimulating a positive response or promoting growth in an animal. Biologically active compositions, complexes or compounds may be used in veterinary applications and compositions.

The term “biologically active” indicates that the composition, complex or compound has an activity that impacts growth, survival or welfare of an animal in a positive sense, impacts an animal suffering from a disease or disorder in a positive sense and/or impacts a pest, pathogen or parasite in a negative sense. Thus, a biologically active composition, complex or compound may cause or promote a biological or biochemical activity within an animal that is detrimental to the growth and/or maintenance of a pest, pathogen or parasite; or of cells, tissues or organs of an animal that have abnormal growth or biochemical characteristics and/or a pest, a pathogen or a parasite that causes a disease or disorder within a host such as an animal.

The term “pest” is defined herein as encompassing vectors of plant, humans or livestock disease, unwanted species of bacteria, fungi, viruses, insects, nematodes mites, ticks or any organism causing harm during or otherwise interfering with the production, processing, storage, transport or marketing of food, agricultural commodities, wood and wood products or animal feedstuffs.

The term “subject” can be any singular or plural subject, including, but not limited to humans and animals, e.g. mammals or birds, specifically from horses, poultry, pigs, cattle, rodents and pets. Said subjects can be healthy subjects or any subjects suffering or going to suffer from an disease from a pest, pathogen, or parasite.

The term “animal,” as used herein, refers to both human and non-human animals, including, but not limited to, mammals, birds and fish, and encompasses domestic, farm, zoo and wild animals, such as, for example, cows, pigs, horses, goats, sheep or other hoofed animals, dogs, cats, chickens, ducks, non-human primates, guinea pigs, rabbits, ferrets, rats, hamsters and mice.

As used herein the terms “cellular organism” “microorganism” or “microbe” should be taken broadly. These terms are used interchangeably and include, but are not limited to, the two prokaryotic domains, Bacteria and Archaea, as well as certain eukaryotic fungi and protists.

The term “prokaryotes” is art recognized and refers to cells that contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mvcobacteria, Micrococcus, others) (2) low G+C group (Bacillus. Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

A “eukaryote” is any organism whose cells contain a nucleus and other organelles enclosed within membranes. Eukaryotes belong to the taxon Eukaiya or Eukaryota. The defining feature that sets eukaryotic cells apart from prokaryotic cells (the aforementioned Bacteria and Archaea) is that they have membrane-bound organelles, especially the nucleus, which contains the genetic material, and is enclosed by the nuclear envelope.

The term “wild-type microorganism” or “wild-type host cell” describes a cell that occurs in nature, i.e. a cell that has not been genetically modified. In the disclosure, “wild type strain” or “wild strain” or “wild type cell line” refers to a cell strain/line that can produce minicells. In some embodiments, wild type bacterial strains and/or cell lines such as E. coli strain p678-54 and B. subtilis strain CU403 can make miniature cells deficient in DNA. Methods for producing such minicells are known in the art. See, for example, Adler et al., 1967, Proc. Natl. Acad. Sci. USA 57:321-326; Inselburg. J, 1970 J. Bacteriol. 102(3):642-647; Frazer 1975, Curr. Topics Microbial. Immunol. 69:1-84, Reeve et al 1973, J. Bacterial. 114(2):860-873; and Mendelson et al 1974 J. Bacteriol. 117(3):1312-1319.

As used herein, the term “nucleic acid” refers to a polymeric form of nucleotides of any length, either ribonucleotides or deoxyribonucleotides, or analogs thereof This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid,” “nucleotide,” and “polynucleotide” are used interchangeably.

As used herein, the term “protease-deficient strain” refers to a strain that is deficient in one or more endogenous proteases. For example, protease deficiency can be created by deleting, removing, knock-out, silencing, suppressing, or otherwise downregulating at lease on endogenous protease. Said proteases can include catastrophic proteases. For example, BL21 (DE3) E. coli strain is deficient in proteases Lon and OmpT. E. coli strain has cytoplasmic proteases and membrane proteases that can significantly decrease protein production and localization to the membrane. In some embodiments, a protease-deficient strain can maximize production and localization of a protein of interest to the membrane of the cell. “Protease-deficient” can be interchangeably used as “protease-free” in the present disclosure.

As used herein, the term “ribonuclease-deficient strain” refers to a strain that is deficient in one or more endogenous ribonuclease. For example, ribonuclease deficiency can be created by deleting, removing, knock-out, silencing, suppressing, or otherwise downregulating at lease on endogenous ribonuclease. Said ribonuclease can include ribonuclease III. For example, HT115 E. coli strain is deficient in RNase III. In some embodiments, a ribonuclease-deficient strain is unable to and/or has a reduced capability of recognizing dsRNA and cleaving it at specific targeted locations. “Ribonuclease-deficient” can be interchangeably used as “ribonuclease-free” in the present disclosure.

As used herein, the term “anucleated cell” refers to a cell that lacks a nucleus and also lacks chromosomal DNA and which can also be termed as an “anucleate cell”. Because eubacterial and archaebacterial cells, unlike eukaryotic cells, naturally do not have a nucleus (a distinct organelle that contains chromosomes), these non-eukaryotic cells are of course more accurately described as being “without chromosomes” or “achromosomal.” Nonetheless, those skilled in the art often use the term “anucleated” when referring to bacterial minicells in addition to other eukaryotic minicells. Accordingly, in the present disclosure, the term “minicells” encompasses derivatives of eubacterial cells that lack a chromosome; derivatives of archaebacterial cells that lack their chromosome(s), and anucleate derivatives of eukaryotic cells that lack a nucleus and consequently a chromosome. Thus, in the present disclosure, “anucleated cell” or “anucleate cell” can be interchangeably used with the term “achromosomal cell.”

As used herein, “carrier,” “acceptable carrier,” or “biologically actively acceptable carrier” refers to a diluent, adjuvant, excipient, or vehicle with which a composition can be administered to its target, which does not detrimentally effect the composition.

As used herein, “plants” and “plant derivatives” can refer to any portion of a growing plant, including the roots, stems, stalks, leaves, branches, seeds, flowers, fruits, and the like. For example, cinnamon essential oil can be derived from the leaves or bark of a cinnamon plant.

As used herein, the term “essential oils” refers to aromatic, volatile liquids extracted from plant material. Essential oils are often concentrated hydrophobic liquids containing volatile aroma compounds. Essential oil chemical constituents can fall within general classes, such as terpenes (e.g., p-Cymene, limonene, sabinene, a-pinene, y-terpinene, b-caryophyllene), terpenoids (e.g., geraniol, citronellal, thymol, carvacrol, carvone, borneol) and phenylpropanoids (e.g., cinnamaldehyde, eugenol, vanillin, safrole). Essential oils can be natural (i.e., derived from plants), or synthetic.

As used herein, the term “essential oil” encompasses within the scope of the present disclosure also botanical oils and lipids. Non-limiting examples of essential oils are sesame oil, pyrethrum (extract), glycerol-derived lipids or glycerol fatty acid derivatives, cinnamon oil, cedar oil, clove oil, geranium oil, lemongrass oil, angelica oil, mint oil, turmeric oil, wintergreen oil, rosemary oil, anise oil, cardamom oil, caraway oil, chamomile oil, coriander oil, guaiacwood oil, cumin oil, dill oil, mint oil, parsley oil, basil oil, camphor oil, cananga oil, citronella oil, eucalyptus oil, fennel oil, ginger oil, copaiba balsam oil, perilla oil, cedarwood oil, jasmine oil, palmarosa sofia oil, western mint oil, star anis oil, tuberose oil, neroli oil, tolu balsam oil, patchouli oil, palmarosa oil, Chamaecyparis obtusa oil, Hiba oil, sandalwood oil, petitgrain oil, bay oil, vetivert oil, bergamot oil, Peru balsam oil, bois de rose oil, grapefruit oil, lemon oil, mandarin oil, orange oil, oregano oil, lavender oil, Lindera oil, pine needle oil, pepper oil, rose oil, iris oil, sweet orange oil, tangerine oil, tea tree oil, tea seed oil, thyme oil, thymol oil, garlic oil, peppermint oil, onion oil, linaloe oil, Japanese mint oil, spearmint oil, giant knotweed extract, and others as disclosed herein throughout.

As used herein, the term “stabilize” or “stabilizing” when used with respect to an active agent, an active ingredients, a composition, a compound, or a formulation refers to prevention of chemical or biological degradation of the active agent in thermal or pH change. In some embodiments, “stabilize” or “stabilizing” includes prevention of pH-driven chemical degradation of an active agent and prevention of temperature-driven degradation of an active aunt. In further embodiments, “stabilize” or “stabilizing” includes prevention against oxidative stress/oxidation, hydrolysis, and any other form of chemical degradation.

The term “bioavailability” includes, generally, the degree to which an active agent, an active ingredient, a drug or other substance becomes available to a target subject after delivery, application or administration. In some embodiments, the term “bioavailability” refers to effective dose of an active agent that reaches intended target or subject.

The term “depletion flocculation” refers to that depletion forces destabilize colloids and bring the dispersed particles together resulting in flocculation. The particles are no longer dispersed in the liquid but concentrated in floc formations. In some embodiments, an active agent taught herein is preserved from depletion flocculation in acidic condition by minicells taught herein.

The preset disclosure provides a novel minicell platform using AgriCell technology, which is a highly modular and tunable biological microcapsule that can encapsulate, stabilize, and effectively deliver a sustained release of active ingredients to promote animal health. The key to the AgriCell technology is that it harnesses the capabilities of synthetic biology to produce a bioencapsulation technology that is environmentally compatible, modular in its functionality, and scalable for agricultural applications. Active, non-pathogenic microbial cells are engineered to produce a bioparticle through asymmetric cell division. These bioparticles are small (about 0.5 μm in diameter), spherical versions of their parent microbial cells and they maintain the properties of the parent cell with one major difference: they lack chromosomal DNA. Therefore, the biological particles retain the benefits of the parent microbe, but do not risk contaminating the environment with modified DNA or outcompeting native species since they do not propagate.

Also, the present disclosure a novel minicell platform using AgriCell technology, which represents a potential platform which not only acts as potent candidate vaccines but also provide a tool for efficient adjuvant and vaccine delivery systems. The major goals of veterinary carriers are to improve the health and welfare of companion animals, to increase production of livestock and aquaculture in a cost-effective manner and to prevent animal-to-human transmission from both domestic animals and wildlife population. Vaccination remains the most efficacious tool to control infectious diseases. Traditionally, live attenuated and killed microorganisms have been used to induce protective immune response against a disease. The live organisms are usually attenuated either by serial passaging in cell culture or the selective disabling of gene associated with pathogenesis and/or survival of the pathogen (Hajam et al. 2017). Unfortunately, during this inactivation process most of the essential structural and immunogenic components of microorganisms are denatured resulting in impaired function and non-efficient immune responses. Thus, killed vaccines generally induce low cell-mediated immune (CMI) responses and shorter duration of immunity as opposed to live vaccines. In accordance with this notion, newer vaccines such as DNA and subunit vaccines have been extensively tried over the last two decades, so far with only limited success. These next generation vaccines, however, are poorly immunogenic in nature as compared to traditional vaccines, and therefore necessitate an appropriate adjuvant in the vaccine formulation. Furthermore, DNA vaccines are not effectively targeted to the antigen presenting cells (APC) and are not presented properly in the context of appropriate danger signals. Therefore, DNA-based vaccines need a better delivery system to reach their full potential (Bergmann-Leitner et al 2013, Ebensen et al. 2004, Brun et al. 2011).

In some embodiments, the minicells taught herein are naturally occurring anucleate cells.

In other embodiments, the present disclose teaches novel minicells that are engineered to encapsulate high-payload capacities of active ingredients for animal health applications. Also, the robustness of minicell production is improved.

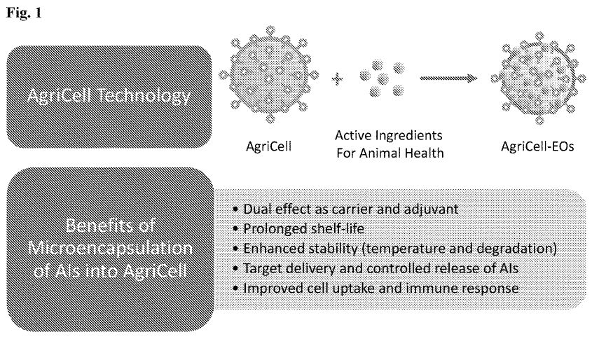

The present disclosure teaches that microencapsulation of active agents/ingredients into AgriCell has two functions: (1) to enhance the oxidative stability, thermo stability, photo stability, shelf-life, and biological activity of phytogenic additives, including the essential oils; and (2) to ensure their targeted delivery in feed to the lower intestine of animals. A schematic view of benefits of using AgriCell microencapsulation strategies is shown in FIG. 1 to overcome limitations of active agents, especially essential oils, as feed additives such as (i) EOs are affected by environmental conditions (light, temperature, or moisture); (ii) high reactivity and volatility; (iii) interaction with food matrix; (iv) low bioavailability and stability, and (v) strong odor and taste.

The present disclosure further provides that the AgriCell as minicells taught herein, serves as a carrier that protects active agents/ingredients from environmental stresses until it delivers its high-payload capacity slowly to a subject through the natural breakdown of its biodegradable membrane. This bio-encapsulation technology overcomes many of the problems of active agent delivery and can serve as the much-needed replacement to traditional techniques using plastic microcapsules.

In some embodiments, the AgriCell technology can be engineered in various ways to improve stability of active agents (such as essential oils) encapsulated into the AgriCell and provide tailored controlled release profiles of the active agents.

In other embodiments, the AgriCell technology can also be genetically engineered in various ways to enhance immune responses against envelope bound antigens, including T-cell activation and mucosal immunity. The advantages of AgriCell platform include the simplicity of the production method, safety, independence from the cold chain, and versatility as a combination vaccine.

The present disclosure teaches successful encapsulation of different active ingredients of interest into minicells as the AgriCell platform for animal health applications, shows outstanding biological activity, improved stability and controlled release.

Table 1 summarizes some of the active ingredients successfully encapsulated by the AgriCell platform, indicating its biological effect and common drawbacks overcome by AgriCell encapsulation.

TABLE 1

| | Active | | |
|---|---|---|---|
| Group | Ingredient | Biological Effect | Common Drawbacks |
| Nucleic | Double- | Activation of | Nucleic acid-based vaccines |
| acids | stranded | dendritic cells, | are not effectively targeted |
| | RNA | production of | to the antigen presenting |
| | (ds-RNA) | immunomodulatory | cells (APC), they need a |
| | | cytokines. | better delivery system to |
| | | | reach their full potential. |
| Anti- | Genistein | A naturally | Poor solubility, absorption, |
| micro- | | occurring | bioavailability and limited |
| bials | | isoflavone found in | clinical efficacy due to |
| | | soybeans, blocks | its low water solubility. |
| | | the invasion of | |
| | | pathogenic bacteria | |
| | | in mammalian | |
| | | epithelial cells. | |
| | Eugenol | A triterpene | Limited water solubility |
| | | glycoside found | and volatility of eugenol |
| | | in clove oil, acts | significantly reduce it |
| | | by disruption of | biologicalefficacy. |
| | | cytoplasmatic | Encapsulation can be used |
| | | bacterial membrane | to prevent early degradation, |
| | | which increases | absorption and improve |
| | | membrane | efficacy and bioavailability. |
| | | nonspecific | |
| | | permeability | |
| | | and affects the | |
| | | transport of ions | |
| | | and ATP. | |
| Proteins | Hen | Activation and | Low cell uptake rates, |
| | egg-white | maturation of | depicting in delayed |
| | lysozyme | dendritic cells, | immune response. |
| | (HEL) | induction of | |
| | | proinflammatory | |
| | | cytokines | |

### Minicells

Minicells are the result of aberrant, asymmetric cell division, and contain membranes, peptidoglycan, ribosomes, RNA, protein, and often plasmids but no chromosome. (Frazer AC and Curtiss III, Production, Properties and Utility of Bacterial Minicells, Curr. Top. Microbial. Immunol. 69:1-84 (1975)). Because minicells lack chromosomal DNA, minicells cannot divide or grow, but they can continue other cellular processes, such as ATP synthesis, replication and transcription of plasmid DNA, and translation of mRNA. Although chromosomes do not segregate into minicells, extrachromosomal and/or episomal genetic expression elements may segregate, or may be introduced into minicells after segregation from parent cells.

In some embodiments, the minicells described herein are naturally occurring.

In other embodiments, the minicells described herein are non-naturally occurring.

Eubacterial Minicells

One type of minicell is a eubacterial minicell. For reviews of eubacterial cell cycle and division processes, see Rothfield et al., Annu. Rev. Genet., 33:423-48, 1999; Jacobs et al., Proc. Natl. Acad. Sci. USA, 96:5891-5893, May, 1999; Koch, Appl. and Envir. Microb., Vol. 66, No. 9, pp. 3657-3663; Bouche and Pichoff, Mol Microbiol, 1998. 29: 19-26; Khachatourians et al., J Bacteriol, 1973. 116: 226-229; Cooper, Res Microbiol, 1990. 141: 17-29; and Danachie and Robinson, “Cell Division: Parameter Values and the Process,” in: Escherichia Coli and Salmonella Typhimurium: Cellular and Molecular Biology, Neidhardt, Frederick C., Editor in Chief, American Society for Microbiology, Washington, D.C., 1987, Volume 2, pages 1578-1592, and references cited therein; and Lutkenhaus et al., “Cell Division,” Chapter 101 in: Escherichia coli and Salmonella typhimurium: Cellular and Molecular Biology, 2^{nd} Ed., Neidhardt, Frederick C., Editor in Chief, American Society for Microbiology, Washington, D.C., 1996, Volume 2, pages 1615-1626, and references cited therein. When DNA replication and/or chromosomal partitioning is altered, membrane-bounded vesicles “pinch off” from parent cells before transfer of chromosomal DNA is completed. As a result of this type of dysfunctional division, minicells are produced which contain an intact outer membrane, inner membrane, cell wall, and all of the cytoplasm components but do not contain chromosomal DNA.

In some embodiments, the bacterially-derived minicells are produced from a strain, including, but are not limited to a strain of Escherichia coli, Bacillus spp., Salmonella spp., Listeria spp., Mycobacterium spp., Shigella spp., or Yersinia spp. In some embodiments, the bacterially-derived minicells are produced from a strain that naturally produces minicells. Such natural minicell producing strains produce minicells, for example, at a 2:1 ratio (2 bacterial cells for every one minicell). In certain embodiments, exemplary bacterial strains that naturally produce minicells include, but are not limited to E. coli strain number P678-54, Coli Genetic Stock Center (CGSC) number: 4928 and B. subtilis strain CU403.

As one example, mutations in B. subtilis smc genes result in the production of minicells (Britton et al., 1998, Genes and Dev. 12:1254-1259; Moriya et al., 1998, Mol Microbiol 29:179-87). Disruption of smc genes in various cells is predicted to result in minicell production therefrom.

As another example, mutations in the divIVA gene of Bacillus subtilis results in minicell production. When expressed in E. coli, B. subtilis or yeast Schizosaccharomyces pombe, a DivIVA-GFP protein is targeted to cell division sites therein, even though clear homologs of DivIVA do not seem to exist in E. coli, B. subtilis or S. pombe (David et al., 2000, EMBO J. 19:2719-2727. Over- or under-expression of B. subtilis DivIVA or a homolog thereof may be used to reduce minicell production in a variety of cells.

In some embodiments, the minicell-producing bacteria is a Gram-negative bacteria. The Gram-negative bacteria includes, but is not limited to, Escherichia coli, Salmonella spp. including Salmonella typhimurium, Shigella spp. including Shigella flexneri, Pseudomonas aeruginosa, Agrobacterium, Campylobacter jejuni, Lactobacillus spp., Neisseria gonorrhoeae, and Legionella pneumophila. In some embodiments, the minicell-producing gram-negative bacteria can produce minicells naturally caused by endogenous or exogenous mutation(s) associated with cell division and/or chromosomal partitioning. In some embodiments, the minicell-producing bacteria comprises endogenous or exogenous gene(s) that is involved in cell division and/or chromosomal partitioning, where the gene is genetically modified such as by homologous recombination, compared to a corresponding wild-type gene. In some embodiments, the minicell-producing grain-negative bacteria is deficient in protease and/or its activity naturally and/or by genetic engineering techniques disclosed herein. In some embodiments, the protease-deficient minicell-producing gram-negative bacteria comprises a recombinant expression vector comprising a gene or genes that is involved in a protein of interest disclosed in the present disclosure.

In some embodiments, the minicell-producing bacteria can be a Gram-positive bacteria. The Grain-positive bacteria includes, but is not limited to, Bacillus subtilis, Bacillus cereus, Corynebacterium Glutamicum, Lactobacillus acidophilus, Staphylococcus spp., or Streptococcus spp. In some embodiments, the minicell-producing gram-positive bacteria can produce minicells naturally caused by endogenous or exogenous mutation(s) associated with cell division and/or chromosomal partitioning. In some embodiments, the minicell-producing gram-positive bacteria comprises endogenous or exogenous gene(s) that is involved in cell division and/or chromosomal partitioning, where the gene is genetically modified such as by homologous recombination, compared to a corresponding wild-type gene. In some embodiments, the minicell-producing gram-positive bacteria is deficient in protease and/or its activity naturally and/or by genetic engineering techniques disclosed herein. In some embodiments, the protease-deficient minicell-producing grain-positive bacteria comprises a recombinant expression vector comprising a gene or genes that is involved in a protein of interest disclosed in the present disclosure.

The minicell-producing bacteria can be a Extremophilic bacteria. The Extremophilic bacteria includes, but is not limited to, Thermophiles including Thermus aquaticus, Psychrophiles, Piezophiles, Halophilic bacteria, Acidophile, Alkaliphile, Anaerobe, Lithoautotroph, Oligotroph, Metallotolerant, Oligotroph, Xerophil or Polyextremophile. In some embodiments, the minicell-producing Extremophilic bacteria can produce minicells naturally caused by endogenous or exogenous mutation(s) associated with cell division and/or chromosomal partitioning. In some embodiments, the minicell-producing Extremophilic bacteria comprises endogenous or exogenous gene(s) that is involved in cell division and/or chromosomal partitioning, where the gene is genetically modified such as by homologous recombination, compared to a corresponding wild-type gene. In some embodiments, the minicell-producing Extremophilic bacteria is deficient in protease and/or its activity naturally and/or by genetic engineering techniques disclosed herein. In some embodiments, the protease-deficient minicell-producing Extremophilic bacteria comprises a recombinant expression vector comprising a gene or genes that is involved in a protein of interest disclosed in the present disclosure.

Eukaryotic Minicells

Achromosomal eukaryotic minicells (i.e., anucleate cells) are within the scope of the disclosure. Yeast cells are used to generate fungal minicells. See, e.g., Lee et al., Ibd1p, a possible spindle pole body associated protein, regulates nuclear division and bud separation in Saccharomyces cerevisiae, Biochim Biophys Acta 3:239-253, 1999; Kopecka et al., A method of isolating anucleate yeast protoplasts unable to synthesize the glucan fibrillar component of the wall J Gen Microbiol 81:111-120, 1974; and Yoo et al., Fission yeast Hrp1, a chromodomain ATPase, is required for proper chromosome segregation and its overexpression interferes with chromatin condensation, Nucl Acids Res 28:2004-2011, 2000. Cell division in yeast is reviewed by Gould and Simanis, The control of septum formation in fission yeast, Genes & Dev 11:2939-51, 1997).

In some embodiments, the eukaryotic minicells can be produced from yeast cells, such as Saccharomyces cerevisiae, Pichia pastoris and/or Schizosaccharomyces pombe.

As one example, mutations in the yeast genes encoding TRF topoisomerases result in the production of minicells, and a human homolog of yeast TRF genes has been stated to exist (Castano et al., 1996, Nucleic Acids Res 24:2404-10). Mutations in a yeast chromodomain ATPase, Hrp1, result in abnormal chromosomal segregation; (Yoo et al., 2000 Nuc. Acids Res. 28:2004-2011). Disruption of TRF and/or Hrp1 function is predicted to cause minicell production in various cells. Genes involved in septum formation in fission yeast (see, e.g., Gould et al., 1997 Genes and Dev. 11:2939-2951) can be used in like fashion.

Platelets are a non-limiting example of eukaryotic minicells. Platelets are anucleate cells with little or no capacity for de novo protein synthesis. The tight regulation of protein synthesis in platelets (Smith et al., 1999, Vasc Med 4:165-72) may allow for the over-production of exogenous proteins and, at the same time, under-production of endogenous proteins. Thrombin-activated expression elements such as those that are associated with Bcl-3 (Weyrich et al., Signal-dependent translation of a regulatory protein, Bcl-3, in activated human platelets, Cel Biology 95:5556-5561, 1998) may be used to modulate the expresion of exogneous genes in platelets.

As another non-limiting example, eukaryotic minicells are generated from tumor cell lines (Gyongyossy-Issa and Khachatourians, Tumour minicells: single, large vesicles released from cultured mastocytoma cells (1985) Tissue Cell 17:801-809; Melton, Cell fusion-induced mouse neuroblastomas HPRT revertants with variant enzyme and elevated HPRT protein levels (1981) Somatic Cell Genet. 7: 331-344).

Yeast cells are used to generate fungal minicells. See, e.g., Lee et al., Ibd1p, a possible spindle pole body associated protein, regulates nuclear division and bud separation in Saccharomyces cerevisiae, Biochim Biophys Acta 3:239-253, 1999; Kopecka et al., A method of isolating anucleate yeast protoplasts unable to synthesize the glucan fibrillar component of the wall J Gen Microbiol 81:111-120, 1974; and Yoo et al., Fission yeast Hrp1, a chromodomain ATPase, is required for proper chromosome segregation and its overexpression interferes with chromatin condensation, Nucl Acids Res 28:2004-2011, 2000. Cell division in yeast is reviewed by Gould and Simanis, The control of septum formation in fission yeast, Genes & Dev 11:2939-51, 1997). In some embodiments, the present disclosure teaches production of yeast minicells.

Archaebacterial Minicells

The term “archaebacterium” is defined as is used in the art and includes extreme thermophiles and other Archaea (Woese, C. R., L. Magrum. G. Fox. 1978. Archaebacteria. Journal of Molecular Evolution. 11:245-252). Three types of Archaebacteria are halophiles, thermophiles and methanogens. By physiological definition, the Archaea (informally, archaes) are single-cell extreme thermophiles (including thermoacidophiles), sulfate reducers, methanogens, and extreme halophiles. The thermophilic members of the Archaea include the most thermophilic organisms cultivated in the laboratory. The aerobic thermophiles are also acidophilic; they oxidize sulfur in their environment to sulfuric acid. The extreme halophiles are aerobic or microaerophilic and include the most salt tolerant organisms known. The sulfate-reducing Archaea reduce sulfate to sulfide in extreme environment. Methanogens are strict anaerobes, yet they gave rise to at least two separate aerobic groups: the halophiles and a thermoacidophilic lineage. Non-limiting examples of halophiles include Halobacterium cutirubrum and Halogerax mediterranei. Non-limiting examples of methanogens include Methanococcus voltae; Methanococcus vanniela; Methanobacterium thermoautotrophicum; Methanococcus voltae; Methanothermus fervidus; and Methanosarcina barkeri. Non-limiting examples of thermophiles include Azotobacter vinelandii; Thermoplasma acidophilum; Pyrococcus horikoshii; Pyrococcus furiosus; and Crenarchaeota (extremely thermophilic archaebacteria) species such as Sulfolobus solfataricus and Sulfolobus acidocaldarius.

Archaebacterial minicells are within the scope of the disclosure. Archaebacteria have homologs of eubacterial minicell genes and proteins, such as the MinD polypeptide from Pyrococcus furiosus (Hayashi et al., EMBO J. 20:1819-28, 2001). It is thus possible to create Archaebacterial minicells by methods such as, by way of non-limiting example, overexpressing the product of a min gene isolated from a prokaryote or an archaebacterium; or by disrupting expression of a min gene in an archaebacterium of interest by, e.g., the introduction of mutations thereof or antisense molecules thereto. See, Laurence et al., Genetics 152:1315-1323, 1999.

By physiological definition, the Archaea (informally, archaes) are single-cell extreme thermophiles (including thermoacidophiles), sulfate reducers, methanogens, and extreme halophiles. The thermophilic members of the Archaea include the most thermophilic organisms cultivated in the laboratory. The aerobic thermophiles are also acidophilic; they oxidize sulfur in their environment to sulfuric acid. The extreme halophiles are aerobic or microaerophilic and include the most salt tolerant organisms known. The sulfate-reducing Archaea reduce sulfate to sulfide in extreme environment. Methanogens are strict anaerobes, yet they gave rise to at least two separate aerobic groups: the halophiles and a thermoacidophilic lineage. In some embodiments, the present disclosure teaches production of archaeal minicells.

### Bacterial Minicell Production

Minicells are produced by parent cells having a mutation in, and/or overexpressing, or under expressing a gene involved in cell division and/or chromosomal partitioning, or from parent cells that have been exposed to certain conditions, that result in aberrant fission of bacterial cells and/or partitioning in abnormal chromosomal segregation during cellular fission (division). The term “parent cells” or “parental cells” refers to the cells from which minicells are produced. Minicells, most of which lack chromosomal DNA (Mulder et al., Mol Gen Genet, 221: 87-93, 1990), are generally, but need not be, smaller than their parent cells. Typically, minicells produced from E. coli cells are generally spherical in shape and are about 0.1 to about 0.3 μm in diameter, whereas whole E. coli cells are about from about 1 to about 3 μm in diameter and from about 2 to about 10 μm in length. Micrographs of E. coli cells and minicells that have been stained with DAPI (4:6-diamidino-z-phenylindole), a compound that binds to DNA, show that the minicells do not stain while the parent E. coli are brightly stained. Such micrographs demonstrate the lack of chromosomal DNA in minicells. (Mulder et al., Mol. Gen. Genet. 221:87-93, 1990).

Minicells are achromosomal, membrane-encapsulated biological nanoparticles (≤1 μm) that are formed by bacteria following a disruption in the normal division apparatus of bacterial cells. In essence, minicells are small, metabolically active replicas of normal bacterial cells with the exception that they contain no chromosomal DNA and as such, are non-dividing and non-viable. Although minicells do not contain chromosomal DNA, the ability of plasmids, RNA, native and/or recombinantly expressed proteins, and other metabolites have all been shown to segregate into minicells. Some methods of construction of minicell-producing bacterial strains are discussed in detail in U.S. patent application Ser. No. 10/154,951(US Publication No. US/2003/0194798 A1), which is hereby incorporated by reference in its entirety.

Disruptions in the coordination between chromosome replication and cell division lead to minicell formation from the polar region of most rod-shaped prokaryotes. Disruption of the coordination between chromosome replication and cell division can be facilitated through the overexpression of some of the genes involved in septum formation and binary fission. Alternatively, minicells can be produced in strains that harbor mutations in genes that modulate septum formation and binary fission. Impaired chromosome segregation mechanisms can also lead to minicell formation as has been shown in many different prokaryotes.

A description of methods of making, producing, and purifying bacterial minicells can be found, for example, in International Patent application No. WO2018/201160, WO2018/201161, and WO2019/060903, which are incorporated herein by reference.

Also, a description of strains for producing minicells an be found, for example, in International Patent application No. WO2018/201160, WO2018/201161, and WO2019/060903, which are incorporated herein by reference.

In some embodiments, the present disclosure teaches a composition comprising: a minicell and an active agent. In some embodiments, the minicell is derived from a bacterial cell. In some embodiments, the minicell is less than or equal to 1 μm in diameter. The minicell is about 10 nm-about 1000 nm in size, about 20 nm-about 900 nm in size, about 30 nm-about 800 nm in size, about 400 nm-about 700 nm in size, about 50 nm-about 600 rim in size, about 60 nm-about 500 nm in size, about 70 nm-about 550 nm in size, about 80 nm-about 500 nm in size, about 90 nm-about 450 nm in size, about 100 nm-about 400 nm in size, and about 100 nm-about 300 nm in size. In other embodiments, the minicell is about 100 nm-about 300 nm in size.

### Active Agents/Ingredients

According to the terms of the disclosure, the term “active agent,” in some embodiments, further means “biocontrol agents” and “biological control agents” referring to agents, ingredients, compounds, compositions or formulations which control pests by interference with their ecological status, which also can be referred to as antagonists. Successful biological control reduces the population density of the target pest. In some embodiments, the active agent as a biocontrol agent originates in a biological matter such as a plant species and is effective in the treatment, prevention, amelioration, inhibition, elimination or delaying the onset of at least one of bacterial, fungal, viral, insect, or any other pest infections or infestations and inhibition of spore germination and hyphae growth. In some embodiments, an active is environmentally safe, that it, it is detrimental to the target species, but does not substantially damage other species in a non-specific manner. Furthermore, it is understood that an active agent also encompasses the term “biochemical control agent” or “biochemical control compound”. The active agents as biochemical control agents include, but are not limited to, plant extracts, essential oils derived from plant species, primary and/or secondary plant metabolites, growth regulators, hormones, enzymes, pheromones, allomones and kairomones, which are either naturally occurring or identical to a natural product, that attract, retard, destroy or otherwise exert a pesticidal, antimicrobial, antibacterial, antifungal, antiviral, anti-inflammatory, and antioxidant activity. In other embodiments, active agents refer to biologically active compounds a polypeptide, a protein, a plant extract, an essential oil, a metabolite, a semiochemical, an enzyme, a hormone, a pheromone, and a nucleic acid such as RNA biomolecule including antisense nucleic acid, dsRNA, shRNA, siRNA, miRNA, ribozyme, and aptamer.

In some embodiments, the present disclose teaches a composition comprising: a minicell and an active agent. The active agent is encapsulated by the minicell. The active agent is a biologically active agent. In some embodiments, the biologically active agent is selected from a nucleic acid, a peptide, a protein, an essential oil, and combinations thereof.

In other embodiments, the present disclosure teaches that active agents encapsulated into a minicell platform are nutrients including but not limited to macro and micronutrients, including carbohydrates, fats, fiber, minerals, proteins, vitamins, antioxidants, essential oils, and water. Products including but not limited to nucleic acids, peptides/enzymes, glycinates, carotenoids, organic acids, vitamins, and conjugated linoleic acids.

Essential Oils

Phytobiotics are considered as a first line alternative to antibiotic growth promotors based on their complex bioactivity, mainly due to antimicrobial, antioxidant, and anti-inflammatory properties of plant bioactive compounds. Biological activities of plant metabolites are positively reflected on feed palatability, digestive functions, and animal intestinal microbiome structure, as well as improved production performance in poultry, pigs, and ruminant and aquaculture animals. Most reports are related to phytobiotics growth-promoting effects. In addition, the effects on reproduction, milk, egg, and meat quality parameters have been documented. Evidence has shown that phytobiotics may minimize the environmental impact of the livestock industry by reducing emissions into the atmosphere of ammonia from pig production, and methane from fermentation in the rumen.

Essential oils (EOs) represent a major group of phytobiotics, consisting of a complex mixture of different volatile and non-volatile compounds. Due to their strong aromatic features and bioactivity, EOs have been widely used since ancient times in aromatherapy, as flavor and fragrances in cosmetics and foods, and more recently as pharmaceuticals, natural preservatives, additives, and biopesticides. The bioactivity of EOs depends on their complex mixture of volatile molecules produced by the secondary metabolism of aromatic and medical plants. Terpenoids are known as a major class of EOs components. Among natural compounds, the terpenoids are the largest family of plant secondary metabolites, with over 40,000 different chemical structures described to date. Factors that influence the bioactivity of EOs, regardless of the field of application, are related to plant species, growing conditions, harvest time, and plant chemotype, among others. Due to the volatile and reactive nature of EOs, their effectiveness in animals can be influenced by different conditions during production processes, storage of EOs, and conditions in the gastrointestinal tract of the animals.

Essential oils such as oregano, thyme, and cinnamon have been disclosed in the art primarily as flavoring or odorizing agents. Essential oils have in some instances been disclosed as pharmaceuticals for medical and veterinary uses, yet the efficacy and compatibility of multi-essential oil blends are unpredictable and often undesirable. For example, U.S. Pat. No. 6,106,838 teaches that essential oils of thyme and oregano exhibit antagonist effects when combined for pharmaceutical uses.

Essential oils such as peppermint oil (PO), thyme oil (TO), clove oil (CO), and cinnamon oil (CnO) have been used for their antibacterial, antiviral, anti-inflammatory, antifungal, and antioxidant properties. Terpenoids such as menthol and thymol and phenylpropenes such as eugenol and cinnamaldehyde are components of EOs that mainly influence antibacterial activities. For example, thymol is able to disturb micromembranes by integration of its polar head-groups in lipid bilayers and increase of the intracellular ATP concentration. Eugenol was also found to affect the transport of ions through cellular membranes. Cinnamaldehyde inhibits enzymes associated in cytokine interactions and acts as an ATPase inhibitor.

In some embodiments, terpenes are chemical compounds that are widespread in nature, mainly in plants as constituents of essential oils (EOs). Their building block is the hydrocarbon isoprene (C₅H₈)n.

In some embodiments, examples of terpenes include, but are not limited to citral, pinene, nerol, b-ionone, geraniol, carvacrol, eugenol, carvone, terpeniol, anethole, camphor, menthol, limonene, nerolidol, framesol, phytol, carotene (vitamin A1), squalene, thymol, tocotrienol, perillyl alcohol, bomeol, myrcene, simene, carene, terpenene, and linalool.

Terpenes as constituents of EOs have been found to inhibit the growth of cancerous cells, decrease tumour size, decrease cholesterol levels, and have a biocidal effect on micro-organisms in vitro. Owawunmi, (Letters in Applied Microbiology, 1993, 9(3): 105-108), showed that growth media with more than 0.01% citral reduced the concentration of E. coli, and at 0.08% there was a bactericidal effect. U.S. Pat. No. 5,673,468 describes a terpene formulation, based on pine oil, used as a disinfectant or antiseptic cleaner. U.S. Pat. No. 5,849,956 teaches that a terpene found in rice has antifungal activity. U.S. Pat. No. 5,939,050 describes an oral hygiene antimicrobial product with a combination of 2 or 3 terpenes that showed a synergistic effect. Several U.S. patents (U.S. Pat. Nos. 5,547,677, 5,549,901, 5,618,840, 5,629,021, 5,662,957, 5,700,679, 5,730,989) teach that certain types of oil-in-water emulsions have antimicrobial, adjuvant, and delivery properties.

Terpenes have been found to be effective and nontoxic dietary anti-tumor agents, which act through a variety of mechanisms of action (Crowell et al. Crit. Rev. Oncog., 1994, 5(1): 1-22; Crowell et al. Adv. Exp. Med. Biol., 1996, 401: 131-136). The terpenes geraniol, tocotrienol, perillyl alcohol, b-ionone, and d-limonene, suppress hepatic HMG-CoA reductase activity, a rate limiting step in cholesterol synthesis, and modestly lower cholesterol levels in animals (Elson et al, J. Nutr., 1994, 124: 607-614). D-limonene and geraniol reduced mammary tumors (Elegbede et al. Carcinogenesis, 1984, 5(5): 661-664; Elegbede et al., J. Natl. Cancer Inst., 1986, 76(2): 323-325; Karlson et al. Anticancer Drugs, 1996, 7(4): 422-429) and suppressed the growth of transplanted tumors (Yu et al., J. Agri. Food Chem., 1995, 43: 2144-2147).

Terpenes have also been found to inhibit the in vitro growth of bacteria and fungi (Chaumont et al.), Ann. Pharm. Fr., 1992, 50(3): 156-166; Moleyar et al., Int. J. Food Microbiol, 1992, 16(4): 337-342; and Pattnaik et al. Microbios, 1997, 89(358): 39-46) and some internal and external parasites (Hooser et al., J. Am. Vet. Med. Assoc., 1986, 189(8): 905-908). Geraniol was found to inhibit growth of Candida albicans and Saccharomyces cerevisiae strains by enhancing the rate of potassium leakage and disrupting membrane fluidity (Bard et al., Lipids, 1998, 23(6): 534-538). B-ionone has antifungal activity which was determined by inhibition of spore germination, and growth inhibition in agar (Mikhlin et al., A. Priki. Biokhim Mikrobiol, 1983, 19: 795-803; Salt et al., Adam. Physiol. Molec. Plant Path, 1986, 28: 287-297). Teprenone geranylgeranylacetone has an antibacterial effect on H. pylori (Ishii, Int. J. Med. Microbiol. Vivol. Parasitol. Infect. Dis., 1993, 280(1-2): 239-243). Rosanol, a commercial product with 1% rose oil, has been shown to inhibit the growth of several bacteria (Pseudomonas, Staphylococus, E. coli, and H. pylori). Geraniol is the active component (75%) of rose oil. Rose oil and geraniol at a concentration of 2 mg/L inhibited the growth of H. pylori in vitro. Some extracts from herbal medicines have been shown to have an inhibitory effect in H. pylori, the most effective being decursinol angelate, decursin, magnolol, berberine, cinnamic acid, decursinol, and gallic acid (Bae et al., Biol. Pharm. Bull., 1998, 21(9) 990-992). Extracts from cashew apple, anacardic acid, and (E)-2-hexenal have shown bactericidal effect against H. pylon.

There may be different modes of action of terpenes against microorganisms; they could (1) interfere with the phospholipid bilayer of the cell membrane, (2) impair a variety of enzyme systems (HMG-reductase), and (3) destroy or inactivate genetic material. It is believed that due to the modes of action of terpenes being so basic, e.g., blocking of cholesterol, that infective agents will not be able to build a resistance to terpenes.

There are, however, a number of drawbacks to the use of terpenes as EOs, such as (i) terpenes are liquids which can make them difficult to handle and unsuitable for certain purposes; (ii) terpenes are not very miscible with water, and it generally requires the use of detergents, surfactants or other emulsifiers to prepare aqueous emulsions, and (iii) terpenes are prone to oxidation in aqueous emulsion systems, which make long term storage a problem.

That is, the main limitations of EOs comprising terpenes and/or terpenoids are their inherent volatility and propensity to oxidize. These drawbacks limit the long-term antibacterial efficacy of EOs.

The present disclosure teaches novel delivery technologies, such as encapsulation using minicells, to protect the volatile compounds and bioactivity of EOs from (1) degradation and oxidation process occurring during feed processing and storage; (2) different conditions in animals' gut and enable the controlled release in the intestinal region of the gut; and (3) mixing with the basal feed constituents.

Eugenol, is a naturally occurring phenol essential oil extracted from cloves, is known to be an antioxidant, a monoamine oxidase (MAO) inhibitor and known to have neuroprotective effects. In addition, eugenol exhibits an excellent bactericidal activity against a wide range of organisms like Escherichia coli, Staphylococcus aureus, Pseudomonas aeruginosa and Listeria monocytogenes (Pandima Devi et al. 2010). The antimicrobial activity of eugenol can be ascribed to the presence of a free hydroxyl group in the molecule conferring strong protein binding properties, preventing enzyme action on bacterial cell membrane. Eugenol first alters membrane permeability, this hyper permeability is followed by leakage of ions and extensive loss of other cellular contents, and ultimately results in cell death. Eugenol has been found to have anti-inflammatory, neuroprotective, antipyretic, antioxidant, antifungal and analgesic properties.

Genistein is one of major isoflavones derived from soybean products and it is believed to have beneficial effects on human and animal health. However, its low water-solubility and poor oral bioavailability severely hamper its use as a functional food ingredient or for veterinary formulations. Genistein blocks the invasion of pathogenic bacteria in mammalian epithelial cells, through a mechanism involving bacterial agglutination and inhibition of internalization properties. Additionally, genistein is used as an antineoplastic and antitumor agent and has antihelmintic activity.

Geraniol is a monoterpene that is found within many essential oils of fruits, vegetables, and herbs including rose oil, citronella, lemongrass, lavender, and other aromatic plants. It is emitted from the flowers of many species of plant and is commonly used by the food, fragrance, and cosmetic industry. Geraniol has demonstrated a wide spectrum of pharmacological activities including antimicrobial, anti-inflammatory, antioxidant, anti-cancer, and neuroprotective. Due to its anticancer effects, geraniol has been found to be effective against a broad range of cancers including breast, lung, colon, prostate, pancreatic, skin, liver, kidney and oral cancers.

Thymol is a phenol that is a natural monoterpene derivative of cymene. It has a role as a volatile oil component. It is a member of phenols and a monoterpenoid. It is used as a stabilizer in pharmaceutic preparations. It has been used for its antiseptic, antibacterial, and antifungal actions.

Pyrethrum is the crude extract form obtained from flowers of the plant Chrysanthemum cinerariifolium. Pyrethrin refers to a more refined extract of pyrethrum. While pyrethrum extract is composed of 6 esters, both organic compounds mediate insecticidal activities. Pyrethrum-containing mixtures are used as a common insecticide to control specific pest species. Pyrethrum extract is also used to treat head, body, and pubic lice infections. The active compound is absorbed by the lice and destroys them by acting on their nervous systems but is thought to exert minimal effect on humans.

In some embodiments, the present disclose teaches a composition comprising: a minicell and an active agent. The active agent is encapsulated by the minicell. The active agent is a biologically active agent. In some embodiments, the biologically active agent is selected from a nucleic acid, a peptide, a protein, an essential oil, and combinations thereof.

In other embodiments, the essential oil comprises geraniol, eugenol, genistein, carvacrol, thymol, pyrethrum or carvacrol.

In some embodiments, the essential oil comprises geraniol.

In some embodiments, the essential oil comprises eugenol.

In some embodiments, the essential oil comprises genistein.

In some embodiments, the essential oil comprises thymol.

In some embodiments, the essential oil comprises pyrethrum.

Essential oils, a major group of phytogenic feed additives (PFA), are considered to be a cost effective and safe alternative to antibiotics as growth promotors. EOs are an important alternative to antibiotics in animal diets. The present disclosure presents that EOs can be used for improvement in the durability of raw feed materials and in the range of positive effects on domestic animals' health and performance.

Due to the high sensitivity of EOs to temperature, pH, and other factors, EOs need to be encapsulated to ensure stability and consistency of the bioactive components of phytobiotics and programmed release in the gastric tract. Evaluation of existent literature data on essential oil stability revealed that oxidative changes and deterioration reactions, which may lead to both sensory as well as pharmacologically relevant alterations, have scarcely been systematically addressed.

In the present disclosure, essential oils are demonstrated. This present disclosure provides compositions including essential oils for enhancing feed efficiency and health of a subject. In some embodiments, subjects include animals. Enhancing feed efficiency can increase the growth rate, weight, weight gain rate, and nutrition of an animal. Enhanced health generally includes one or more of reduced or eliminated microbial infection, reduced or eliminated oxidative stress, reduced or eliminated infection or death during transport, reduced or eliminated microbial infection, increased body weight, enhanced egg characteristics, increased rate of weight gain, increased growth rate, reduced or eliminated birth mortality, increased or improved body score conditions, increased reproductive success, and increased gut health maturation.

Enhanced health can include various benefits specific to a certain class of animals. For example, enhanced health in poultry can include increased growth rate, feed efficiency, egg shell thickness and mortality rate. Enhanced health in dairy cows, or other milk-producing animals such as camels, goats, and sheep, can include increased milk production. In swine, for example, enhanced health can include a reduction or elimination of wasting diseases and mulberry heart.

In the present disclosure, essential oil compositions as described herein can provide antioxidant properties to a host system and generally reduce oxidative stress in animals. Essential oils provided herein can have high oxygen radical absorbance capacity (ORAC), which is the ability of a compound or composition to act as a proton donor and reducing agent for oxygen radicals. While many antioxidants are only effective against single ROS, the essential oils provided herein have high ORAC against peroxyl radicals, hydroxyl radicals, peroxynitrite, superoxide anions, and singlet oxygen, among others. Moreover, essential oils provided herein comprise both lipophilic and hydrophilic characteristics which provide complete cellular protection against ROS. For example, essential oil compositions provided herein can neutralize ROS both in cytoplasm and cell walls.

The essential oil compositions as provided herein can further be used as analogous or substitutes for many commercial products used today, such as antibiotics, growth hormones, immunomodulators, antioxidants, digestive stimulants, and products that increase the performance and quality of animals and other similar products.

Essential oils as provided herein also contain essential oils derived from plants (i.e., “natural” essential oils) and additionally or alternatively their synthetic analogues. Some embodiments comprise a combination of essential oils. Other embodiments comprise a combination of natural and synthetic essential oils. In some embodiments, synthetic essential oils can be a synthetic blend, which generally mimics an essential oil assay of a natural essential oil by including at least 5, at least 10, at least 15, or at least 20 of the most critical essential oils within a natural essential oil.

In some embodiments, the essential oils can include oils from the classes of terpenes, terpenoids, phenylpropenes and combinations thereof.

Protein

A protein active agent may comprise one or more of an amino acid, a polypeptide, a protein or an enzyme. The AgriCell delivery system of the present disclosure may be useful for in vivo or in vitro delivery of active agents, such as, amino acids, peptides and proteins. Peptides can be signaling molecules such as hormones, neurotransmitters or neuromodulators, and can be the active fragments of larger molecules, such as receptors, enzymes or nucleic acid binding proteins. The proteins can be enzymes, structural proteins, signaling proteins or nucleic acid binding proteins, such as transcription factors.

It is widely accepted that T-cell receptors (TcR) usually recognize processed peptide, rather than native peptide, bound to major histocompatibility complex (MHC) molecules. Thus, synthetic peptides can mimic naturally processed antigens for T-cell activation. It is therefore hypothesized that antigen-presenting cells (APC) process native antigen in the form of peptides, probably bound in the groove of MHC molecules where most polymorphic residues are concentrated. One of the best analyzed antigenic systems in mice is the family of lysozymes and their peptides, particularly those of hen egg-white lysozyme (HEL). Macrophages are effector cells of innate immunity and link innate immune responses to acquired immunity through antigen presentation to memory lymphocytes.

In some embodiments, the present disclose teaches a composition comprising: a minicell and an active agent. The active agent is encapsulated by the minicell. The active agent is a biologically active agent. In some embodiments, the biologically active agent is selected from a nucleic acid, a peptide, a protein, an essential oil, and combinations thereof.

In some embodiments, an active agent is a protein including, but not limited to newcastle disease virus HN protein, VP 1 and VP2 capsid proteins, envelope spike (S) protein, S1 glycoprotein, VP60 protein, recombinant E2 protein, PCV2 ORF2 protein, ovalbumin, lysozyme, androstenedione-human serum albumin conjugate and ovandrotone albumin. In some embodiments, the protein is Hen egg-white lysozyme (HEL).

In some embodiments, an active agent is a peptide including, but not limited to anti-luteinizing hormone-releasing hormone (LHRH) peptides, casecidin, isracidin, fertirelin, cecropin, magainin, defensins, protegrin, polymyxin and peptide fragments from virus diseases (foot and mouth disease, FMD vaccine). In other embodiments, an active agent is a synthetic peptide such as gonadotrophin-releasing hormone (GnRh) desiorelin, leuprolide, desmopressin and buserelin, which can be used as a veterinary medicine and in livestock breeding.

Nucleic Acids

A polynucleotide active agent may comprise one or more of an oligonucleotide, an antisense construct, a siRNA, an enzymatic RNA, a recombinant DNA construct, an expression vector, and mixtures thereof. The AgriCell delivery system of the present disclosure may be useful for in vivo or in vitro delivery of nucleic acids.

The present disclose teaches a composition comprising: a minicell and an active agent. The active agent is encapsulated by the minicell. The active agent is a biologically active agent. In some embodiments, the biologically active agent is selected from a nucleic acid, a peptide, a protein, an essential oil, and combinations thereof.

In other embodiments, the nucleic acid is selected from the group consisting of an antisense nucleic acid, a double-stranded RNA (dsRNA), a short-hairpin RNA (shRNA), a small-interfering RNA (siRNA), a microRNA (miRNA), a ribozyme, an aptamer, and combination thereof.

Th present disclosure teaches that the nucleic acid as an active agent can be used for vaccines and therapeutics to treat, cure, or prevent illnesses that affect animals Immunization of animals with naked DNA encoding protective viral antigens would in many ways be an ideal procedure for viral vaccines, as it not only overcomes the safety concerns of live vaccines and vector immunity but also promotes the induction of cytotoxic T cells after intracellular expression of the antigens.

Some examples of nucleic acid-based veterinary vaccines containing specific plasmids or exogenous dsRNAs can be used to treat, cure or prevent diseases caused by surface glycoprotein of IHN virus, West Nile-virus (WNV), feline immunodeficiency virus (FIV), canine parvovirus, rabies virus, feline leukemia virus (FeLV), influenza virus, Aujeszky's disease, porcine respiratory and reproductive syndrome virus, foot-and-mouth disease virus, bovine herpes virus type 1 (BHV-1), bovine respiratory syncytial virus (BRSV) and bovine viral diarrhea virus.

Further examples of nucleic acid-based veterinary vaccines containing specific plasmids or exogenous dsRNAs can be used to treat, cure or prevent diseases caused by from AHS, African horse sickness; AHSV, African horse sickness virus; AIDS, acquired immunodeficiency syndrome; ASF, African swine fever; BCG, Bacille Calmette-Guérin; BSE, Bovine spongiform encephalopathy; BTV, bluetongue virus; CMLV, camelpox virus; CSF, Classical swine fever; CSFV, classical swine fever virus; ELISA, enzyme-linked immunosorbent assay; enJSRV, endogenous JSRV-related retroviruses; F, fusion glycoprotein; FAO, Food and Agriculture Organization; FMD, foot-and-mouth disease; FMDV, foot-and-mouth disease virus; GREP, Global Rinderpest Eradication Program; GTPV, goatpox virus; HA, hemagglutinin; HIV, human immunodeficiency virus; HN, hemagglutinin-neuraminidase; HTLV, human T cell leukemia virus; JSRV, jaagsiekte sheep retrovirus; loNDV, low virulence NDV; MVV, Maedi-visna virus; NA, neuraminidase; NDV, Newcastle disease viruses; OIE, Office International des Epizooties; OPA, ovine pulmonary adenocarcinoma; RSV, Rous sarcoma virus; RT-PCR, reverse transcriptase-polymerase chain reaction; SA, sialic acids; SARS, severe acute respiratory syndrome; SARS-CoV, SARS-Coronavirus; SPY, sheeppox virus; TB, tuberculosis; TSE, transmissible spongiform encephalopathy; VARV, variola virus; vCJD, variant Creutzfeldt-Jakob disease; vNDV, virulent NDV.

The present disclosure also teaches use of minicell platform for a nucleic acid (i.e. dsRNA) to treat, cure or prevent fungal linfections in both human and animals, including the prevention of transmission of fungal pathogens between animals and humans. The different categories of fungal infections can be encountered in animals originating from environmental sources without transmission to humans. In addition, the endemic infections with indirect transmission from the environment, the zoophilic fungal pathogens with near-direct transmission, the zoonotic fungi that can be directly transmitted from animals to humans, mycotoxicoses and antifungal resistance in animals can occur. Opportunistic mycoses are responsible for a wide range of diseases from localized infections fatal disseminated diseases, such as aspergillosis, mucormycosis, candidiasis, cryptococcosis and infections caused by melanized fungi. The amphibian fungal disease chytridiomycosis and the Bat White-nose syndrom are due to obligatory fungal pathogens. Zoonotic agents are naturally transmitted from vertebrate animals to humans and vice versa. The list of zoonotic fungal agents is limited but some species, like Microsporum canis and Sporothrix brasiliensis from cats, have a strong public health impact. Mycotoxins are defined as the chemicals of fungal origin being toxic for warm-blooded vertebrates. Intoxications by aflatoxins and ochratoxins represent a threat for both human and animal health.

In some embodiments, resistance to fungal infections can be obtained in different animal species that receive a nucleic acid (i.e. dsRNA targeting a gene or genes associated with survival or growth of fungi causing the aforementioned fungal infections) via the minicell platform taught herewith.

In some embodiments, an active agent is a double-stranded RNA (dsRNA) that can be detected by pattern recognition receptors, for example, TLR3, MDA-5, NLRP3 to induce proinflammatory cytokines responsible for innate/adaptive immunity.

RNAi is a post-transcription gene regulation mechanism that is present in all known eukaryotes. The cellular RNAi machinery is initiated by dsRNAs that are initially processed into small interfering RNAs (siRNAs) by Dicer-like (DCL) proteins and eventually leads to the degradation of target mRNAs through the action of the gene silencing complex (RISC). RNAi-based genetic transformation technology has widely been utilized to control several insect pests, and diseases, in what is collectively coined as ‘host-induced gene silencing’ (HIGS) (Fire et al 1998; Baulcombe et al 2015). For instance, the expression of dsRNAs targeting dc1/2 or target of the rapamycin (TOR) genes of B. cinerea significantly suppressed gray mold disease progression in transgenic Arabidopsis, potato and tomato plants, respectively (Xiong et al 2019).

The present disclosure teaches the advantage of the minicell platform, which is that the encapsulation capsule and biomolecule of interest, in this case dsRNA, can both be produced in one fermentation batch. Once the dsRNA is produced and encapsulated in the minicell, the dsRNA is significantly more stable than dsRNA on its own. In some embodiments, the minicell platform has proven to significantly enhance the stability of dsRNA.

The present disclosure provides the development and applicability of minicell-based RNAi technology, as a nontoxic alternative to chemical fungicides. The disclosure presents a robust, scalable platform for producing Minicell-encapsulated-dsRNAs (ME-dsRNAs) using a prokaryotic expression system that sufficiently addresses the major shortcomings of exogenous dsRNA delivery; especially those related to stability, efficacy and scalability. Using this platform, inventors were able to produce a minicell-encapsulated dsRNA delivery system, which demonstrated efficacy and specificity of ME-dsRNAs in inhibiting fungal growth. This synthetic biology platform has the potential to be incorporated into commercial disease management programs against B. cinerea and other economically-important phytopathogenic fungi.

The present disclosure teaches the use of AgriCell platform for RNAi technology in integrated pest/disease management programs for controlling pests, viruses, and other fungal pathogens, in a sustainable way.

In some embodiments, bacteria-derived minicells can be utilized as a cost-effective, scalable platform for dsRNA production and encapsulation. A description of methods of making, producing, and purifying bacterial minicells for RNAi (i.e. dsRNAs) can be found, for example, in International Patent application WO2019/060903, which is incorporated herein by reference.

In some embodiments, Minicell-encapsulated dsRNA (ME-dsRNA) is shielded from RNase degradation and stabilized, enabling the persistence of dsRNA in field-like conditions. ME-dsRNAs targeting chitin synthase class III (Chs3a, Chs3b) and DICER-like proteins (DCL1 and DCL2) genes of B. cinerea selectively knocked-down the target genes and led to significant fungal growth inhibition.

In some embodiments, the potential of ME-dsRNAs to enable the commercial application of RNAi based species-specific biocontrol agents that are comparable in efficacy to conventional synthetics. ME-dsRNAs offer a platform that can readily be translated to large-scale production and deployed to control pests.

In other embodiments, ME-dsRNAs can be used to treat or cure cancers in animals; including, but not limited to, (i) cats such as Feline Leukemia and the Feline Leukemia Virus, Squamous Cell Carcinoma, Feline Mammary Cancer and (ii) dogs such as Canine Transmissible Venereal Tumor, Canine Osteosarcoma, Canine Hemangiosarcoma, Canine Mast Cell Tumors.

Nutrients

In some embodiments, the present disclose teaches a composition comprising: a minicell and an active agent. The active agent is encapsulated by the minicell. The active agent is a biologically active agent.

In some embodiments, the active agent is a nutrient including carbohydrates, fats, fiber, minerals, proteins, carbohydrates, fibers, vitamins, antioxidants, essential oils, and water. Examples of key nutrients for animal health can be classified as (i) proteins and amino acids (such as arginine, histidine, isoleucine, leucine, lysine, methionine, phenylalanine, threonine, tryptophan, valine, taurine, collagen and gelatin), (ii) fats (such as triglycerides, omega-3, omega-6, or omega-9 fatty acids, linoleic acid, tocopherols, arachidonic acid, docosahexaenoic acid (DHA), eicosapentaenoic acid (EPA)), (iii) carbohydrates (glucose, galactose, and fructose, lactose, disaccharides and oligosaccharides), (iv) fibers (cellulose and its derivatives, polysaccharides, and glycosaminoglycans), (v) vitamins (A, B-complex, D, C, E, K, thiamine and β-carotene), (vi) minerals (macrominerals such as sodium, potassium, calcium, phosphorus, magnesium), (vii) trace minerals of known importance such as iron, zinc, copper, iodine, fluorine, selenium, chromium, (viii) other minerals useful for animal nutrition such as cobalt, molybdenum, cadmium, arsenic, silicon, vanadium, nickel, lead, tin and (ix) antioxidants such as ascorbic acid, polyphenols, tannins, flavonols and triterpenes glucosides.

Other Agents

In some embodiments, active agent comprises a small organic active agent. It may comprise a therapeutic agent or a diagnostic agent. In particular embodiments, a small organic active agent may comprise a sequence-specific DNA binding oligomer, an oligomer of heterocyclic polyamides, for example, those disclosed in U.S. Pat. No. 6,506,906 which is hereby incorporated by reference. Other small organic active agents may comprise those disclosed in and by Dervan in “Molecular Recognition of DNA by Small Molecules, Bioorganic & Medicinal Chemistry (2001) 9: 2215-2235”, which is hereby incorporated by reference. In certain embodiments, the oligomer may comprise monomeric subunits selected from the group consisting of N-methylimidazole carboxamide, N-methylpyrrole carboxamide, beta-alanine and dimethyl aminopropylamide.

In another embodiment of the present disclosure, the AgriCell delivery system of the present disclosure may include an inorganic active agent, e.g. gastrointestinal therapeutic agents such as aluminium hydroxide, calcium carbonate, magnesium carbonate, sodium carbonate and the like.

### Use of AgriCell Platform for Animal Nutrition and Health

As used herein, the term “AgriCell” refers to a “minicell” taught herein, both of which are interchangeably used.

The present disclosure teaches that the AgriCell platform can be used to effectively deliver nutrients to the intended animal for improved nutrition. Nutrients that cannot be expressed in the host bacterial system can be loaded into “empty” minicells. Once the nutrient is encapsulated in the minicell, the minicell can be processed to improve the strength of its membrane for enhanced delivery and uptake. Encapsulated nutrients can be used to improve feed conversion, animal performance, fertility, breeding performance, bone health, feed preservation, feed cost savings, feed hygiene, pigmentation, udder health, milk yield, hoof health, meat quality, sustainability, vitality, and wellbeing.

The present disclosure provides that AgriCell platform can protect essential oils and ensure their delivery to the lower gastrointestinal tract (GIT). Without proper protection, most orally administrated essential oils may not reach the lower intestine where most foodborne pathogens reside and propagate. In addition, essential oils tend to interact with food or feed components, leading to reduced antimicrobial activity. Consequently, the use of AgriCell platform provides improved performance in terms of chemical stability under gastric conditions, better protection to autoxidative processes during storage, and improving bioavailability through an encapsulation and controlled release mechanism.

In some embodiments, the composition comprising a minicell and an active agent can be used in various target markets including but not limited to poultry, pigs, ruminants, aquaculture, dairy cows, and companion animals. The active agent taught herein encompasses nutrients such as macro and micronutrients including carbohydrates, fats, fiber, minerals, proteins, vitamins, antioxidants, essential oils, and water.

The present disclosure teaches that the AgriCell platform can be applied to encapsulation of active ingredients of interest for animal health. The AgriCell platform, showing improved stability and bioavailability, long lasting shelf-life and controlled release properties, can be used for animal vaccination.

The term “vaccine,” as used herein, refers to a material capable of producing an immune response and can include an essential oil derived from a plant species, a metabolite derived from a plant species, a wild-type protein, a fusion protein, a particle comprising the fusion protein, or combination thereof.

The term “immune response,” as used herein, refers to an alteration in the reactivity of the immune system of a subject in response to an immunogen and may involve antibody production, induction of cell-mediated immunity, complement activation and/or development of immunological tolerance.

The terms “immunization” and “vaccination” are used interchangeably herein to refer to the administration of a vaccine to a subject for the purposes of raising an immune response and can have a prophylactic effect, a therapeutic effect, or a combination thereof. Immunization can be accomplished using various methods depending on the subject to be treated including, but not limited to, intraperitoneal injection (i.p.), intravenous injection (i.v.), intramuscular injection (i.m.), oral administration, spray administration and immersion.

In some embodiments, an essential oil derived from a plant species, a metabolite derived from a plant species, a wild-type protein, a fusion protein, a particle comprising the fusion protein, or combination thereof.

Encapsulation

The present disclosure teaches a composition comprising: a minicell and an active agent. In some embodiments, the active agent is encapsulated by the minicell.

In some embodiments, the minicell and the active agent are present in a weight-to-weight ratio of about 5:1 to about 1:5 in the composition. In some embodiments, the minicell and the active agent are present in a weight-to-weight ratio of about 4:1 to about 1:4 in the composition. In some embodiments, the minicell and the active agent are present in a weight-to-weight ratio of about 3:1 to about 1:3 in the composition. In some embodiments, the minicell and the active agent are present in a weight-to-weight ratio of about 2:1 to about 1:2 in the composition. In some embodiments, the minicell and the active agent are present in a weight-to-weight ratio of about 1:1 in the composition.

In some embodiments, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, of the active agent is encapsulated by the minicell.

In some embodiments, at least about 10% of the active agent is encapsulated by the minicell.

In some embodiments, the minicell stabilizes the active agent in an acidic condition. The acidic condition is less than pH 7, pH 6, pH 5, pH 4, pH 3, or pH 2. In some embodiments, the minicell encapsulating the active agent is preserved from depletion flocculation when a pH is adjusted to an extremely acidic condition. In some embodiments, the acidic condition is as low as pH 1, pH 2, pH 3, pH 4, pH 5, or pH 6.The extremely acidic condition is as low as pH 1.

In other embodiments, the minicell stabilizes the active agent at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, at least 35 days, at least 36 days, at least 37 days, at least 38 days, at least 39 days, at least 40 days, at least 45 days, at least 50 days, at least 55 days, or at least 60 days, at room temperature in a neutral pH condition. In other embodiments, the minicell stabilizes the active agent at least 30 days, at room temperature in a neutral pH condition.

In some embodiments, the minicell stabilizes the active agent in a thermal variation. In some embodiments, the active agent encapsulated by the minicell is at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, at least 5 fold more resistant to thermal degradation than a free active agent not encapsulated by the minicell after a heat treatment. In other embodiments, the heat treatment is above room temperature, which is at 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., or higher.

In other embodiments, the active agent encapsulated by the minicell is at least 1.1 fold more resistant to thermal degradation than a free active agent not encapsulated by the minicell after a heat treatment. In further embodiments, the active agent encapsulated by the minicell is at least 1.1 fold more resistant to thermal degradation than a free active agent not encapsulated by the minicell after a heat treatment on day 7 after a heat treatment at 40° C.

In some embodiments, the active agent encapsulated by the minicell has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, or less than about 10% thermal degradation after a heat treatment.

In some embodiments, the active agent encapsulated by the minicell has less than about 60% thermal degradation after a heat treatment. In some embodiments, the active agent encapsulated by the minicell has less than about 60% thermal degradation on day 7 after a heat treatment at 40° C.

In some embodiments, the minicell protects the active agent from oxidative degradation by ultraviolet (UV) or visible light. In some embodiments, the active agent encapsulated by the minicell is at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, at least 5 fold more resistant to oxidative degradation than a free active aunt not encapsulated by the minicell under UV or visible light exposure.

In other embodiments, the active agent encapsulated by the minicell is at least 1.1 fold more resistant to oxidative degradation than a free active agent not encapsulated by the minicell under UV or visible light exposure. In further embodiments, the active agent encapsulated by the minicell is at least 1.1 fold more resistant to oxidative degradation than a free active agent not encapsulated by the minicell on day 7 under UV or visible light exposure.

In some embodiments, the active agent encapsulated by the minicell has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%,or less than about 10% oxidative degradation under UV or visible light exposure.

In other embodiments, the active agent encapsulated by the minicell has less than about 35% oxidative degradation under UV or visible light exposure. In further embodiments, the active agent encapsulated by the minicell has less than about 35% oxidative degradation on day 7 under UV or visible light exposure.

The present disclosure teaches that the minicell confers to the active agent an improved stability, an enhanced bioavailability and an extended shelf life. The present disclosure teaches a composition comprising the minicell encapsulates the active agent, thereby conferring to an improved stability, an enhanced bioavailability and an extended shelf life.

Release of Active Agents Encapsulated into AgriCell Platform

The present disclosure teaches a composition comprising: a minicell and an active agent. In some embodiments, the active agent is encapsulated by the minicell.

In some embodiments, a release of the active agent encapsulated by the minicell is delayed when compared to a free active agent not encapsulated by the minicell.

In some embodiments, a release percentage (%) of the encapsulated active agent is less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%,or less than about 10% after a release.

In some embodiments, a release percentage (%) of the encapsulated active agent is less than about 50% in a first hour.

In some embodiments, a release percentage (%) of the encapsulated active agent is at least about 45% at 8 hours after the release.

In some embodiments, the encapsulated active agent has an extended release with less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, or less than about 10% of the active agent retained, when compared to the non-encapsulated free active agent that are fully released, at 8 hours after the release.

In some embodiments, the encapsulated active agent has an extended release with less than about 50% of the active agent retained, when compared to the non-encapsulated free active agent that are fully released, at 8 hours after the release.

The present disclosure teaches that the AgriCell platform can be coated by biopolymer. The biopolymer is a chitosan. In some embodiments, the minicell is coated by biopolymer. In some embodiments, the biopolymer is a chitosan.

In some embodiments, a release of the active agent encapsulated by the biopolymer-coated minicell is further delayed when compared to the encapsulated active agent without the biopolymer coated.

In some embodiments, the active agent encapsulated by the biopolymer-coated minicell has a further extended release with at least about 1%, at least about 5%, at least about 10%, at least about 20%, at least about 30%, at least about 40%, at least about 50%, at least about 60%, at least about 70%, at least about 80%, at least about 90% of the active agent retained, when compared to the encapsulated active agent without the biopolymer coated, after a release.

In some embodiments, the active agent encapsulated by the biopolymer-coated minicell has a further extended release with at least about 10% of the active agent retained, when compared to the encapsulated active agent without the biopolymer coated, at 8 hours after the release.

In some embodiments, the active agent encapsulated by the minicell is capable of being delivered to a target in a controlled release manner

Amounts of Active Agents Delivered by AgriCell Platform

In some embodiments, active aunts are encapsulated within the minicells described herein and delivered to a desired subject. Amounts of active agents of interest are provided herein with percent weight proportions of the various components used in the preparation of the minicell for the encapsulation and deliver of active agents.

The percent weight proportions of the various components used in the preparation of the minicell for the encapsulation and deliver of active agents can be varied as required to achieve optimal results. In some embodiments, the active agents including, but are not limited to a nucleic acid, a polypeptide, a protein, an enzyme, an organic acid, an inorganic acid, a metabolite, an essential oil, a nutrient, and a semiochemical, are present in an amount of about 0.1 to about 99.9% by weight, about 1 to about 99% by weight, about 10 to about 90% by weight, about 20 to about 80% by weight, about 30 to about 70% by weight, about 40 to about 60% by weight, based on the total weight of the minicells within which an active compound of interest is encapsulated. Alternate percent weight proportions are also envisioned.

Among the various aspects of the present disclosure is an minicell in the form of encapsulation of an active agent of interest at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, by weight of the active agent within the minicell.

In other embodiments, the active agent within the minicell is present in an amount of at least about 0.01 g/L, at least about 0.02 g/L, at least about 0.03 g/L, at least about 0.04 g/L, at least about 0.05 g/L, at least about 0.06 g/L, at least about 0.07 g/L, at least about 0.08 g/L, at least about 0.09 g/L, at least about 0.1 g/L, at least about 0.2 g/L, at least about 0.3 g/L, at least about 0.4 g/L, at least about 0.5 g/L, at least about 0.6 g/L, at least about 0.7 g/L, at least about 0.8 g/L, at least about 0.9 g/L, at least about 1 g/L, about 2 g/L, at least about 3 g/L, at least about 4 g/L, at least about 5 g/L, at least about 6 g/L, at least about 7 g/L, at least about 8 g/L, at least about 9 g/L, at least about 10 g/L, at least about 11 g/L, at least about 12 g/L, at least about 13 g/L, at least about 14 g/L, at least about 15 g/L, at least about 16 g/L, at least about 17 g/L, at least about 18 g/L, at least about 19 g/L, at least about 20 g/L, at least about 30 g/L, at least about 40 g/L, at least about 50 g/L, at least about 60 g/L, at least about 70 g/L, at least about 80 g/L, at least about 90 g/L, at least about 100 g/L, at least about 110 g/L, at least about 120 g/L, at least about 130 g/L, at least about 140 g/L, at least about 150 g/L, at least about 160 g/L, at least about 170 g/L, at least about 180 g/L, at least about 190 g/L, at least about 200 g/L, at least about 300 g/L, at least about 400 g/L, at least about 500 g/L, at least about 600 g/L, at least about 700 g/L, at least about 800 g/L, at least about 900 g/L, or at least about 1000 g/L.

In another embodiment, the active agent of interest and the minicell are present in compositions of the disclosure in a weight ratio of about 1:200, about 1:195, about 1:190, about 1:185, about 1:180, about 1:175, about 1:170, about 1:165, about 1:160, about 1:155, about 1:150, about 1:145, about 1:140, about 1:135, about 1:130, about 1:125, about 1:120, about 1:115, about 1:110, about 1:105, about 1:100, about 1:95, about 1:90, about 1:85, about 1:80, about 1:75, about 1:70, about 1:65, about 1:60, about 1:55, about 1:50, about 1:45, about 1:40, about 1:35, about 1:30, about 1:25, about 1:20, about 1:15, about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, about 10:1, about 15:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, about 100:1, about 110:1, about 115:1, about 120:1, about 125:1, about 130:1, about 135:1, about 140:1, about 145:1, about 150:1, about 155:1, about 160:1, about 165:1, about 170:1, about 175:1, about 180:1, about 185:1, about 190:1, about 195:1, or about 200:1. In another embodiment, the active agent of interest and the minicell are present in a weight ratio of from about 1:50 to about 50:1, from about 1:40 to about 40:1, from about 1:30 to about 30:1, from about 1:20 to about 20:1, from about 1:10 to about 10:1, from about 1:5 to about 5:1, from about 1:4 to about 4:1, from about 1:3 to about 3:1, from about 1:2 to about 2:1 or from about 1 to about 1.

In some embodiments, an active agent of interest, for example, is present in at least about 1%, at least about 5% at least about 10%, at least about 20%, at least about 30%, at least about 40%, at least about 50%, at least about 60%, at least about 70%, at least about 80%, at least about 90%, or at least about 95% of total mass of a formulated product. In further embodiments, about 10 to 90% of the total mass of the formulated product is provided for the active agent disclosed herein and the remaining about 10 to 90% of the mass is from the minicell.

In some embodiments, more than one non-expressed active agents can be encapsulated within the minicell. In another embodiment, the formulated product comprises two active agents that are present in compositions of the disclosure in a weight ratio of about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, or about 10:1.

Methods of Producing and Delivering Active Agents to a Subject using AgriCell Platform

The present disclosure provides a method of preparing an minicell encapsulating an active agent, said method comprising the steps of: a) producing and purifying minicells; b) providing an active agent; c) loading the minicells with the active agent for encapsulation; and d) recovering the minicells encapsulating the active agent.

In some embodiments, in step a) the minicells are produced from a bacterial cell. In some embodiments, in step a) the purified minicells are provided as a suspension in water or other suitable liquid, or a concentrated paste. In some embodiments, the suspension comprises about 0.01 to 5,000 mg minicells per ml, about 0.1 to 3,000 mg minicells per ml, about 1 to 1,000 mg minicells per ml, or about 1 to 500 mg minicells per ml. In some embodiments, in step a) the purified minicells are provided as a dry powder.

In some embodiments, in step b) the active agent provided as a suspension in an aqueous solvent.

In some embodiments, in step c) the loaded minicells are suspended in a suspension of the active agent. In some embodiments, in step c) the reaction is carried out at atmospheric pressure at a temperature of about 1° C. to about 40° C., about 5° C. to about 40° C., about 10° C. to about 40° C., or about 20° C. to about 40° C. In some embodiments, in step c) the reaction is carried out at atmospheric pressure at a temperature of about 20° C. to about 37° C. In some embodiments, in step c) the loading ratio between the minicells and the active agent is about 1:5 to about 5:1.

In some embodiments, the method described above further comprises the step of drying the minicells encapsulating the active agent. In some embodiments, the drying of the minicells encapsulating the active agent is by evaporating a solvent.

Among the methods of the present disclosure, the active agent is a biologically active agent. In some embodiments, the biologically active agent is selected from a nucleic acid, a peptide, a protein, an essential oil, and combinations thereof. In some embodiments, the nucleic acid is selected from the group consisting of an antisense nucleic acid, a double-stranded RNA (dsRNA), a short-hairpin RNA (shRNA), a small-interfering RNA (siRNA), a microRNA (miRNA), a ribozyme, an aptamer, and combination thereof. In some embodiments, the terpene is geraniol, eugenol, genistein, carvacrol, thymol, pyrethrum or carvacrol.

The present disclosure provides a method of producing an animal feed for enhancing health of an animal. In some embodiments, said method comprises applying to an animal feed a composition that comprises a minicell and an active agent taught herein.

In some embodiments, the animal feed comprises at least one animal feed ingredient selected from the group consisting of a feed carbohydrate a feed protein, a fee vitamin and mixture thereof. In some embodiments, the animal health indicator is selected from the group consisting of: gut lesion formation, gut microbiota, weight change, feed conversion ratio, and life span.

The present disclosure provides a method of enhancing health of an animal, said method comprising: administering to an animal in need thereof an effective amount of a composition that comprises a minicell and an active agent taught herein.

Among the methods of the present disclosure, the health of the animal administered with the composition is enhanced when compared to the health of the animal not administered with the composition. In some embodiments, the composition is administered with an animal feed. In some embodiments, the animal feed comprises at least one animal feed ingredient selected from the group consisting of a feed carbohydrate, a feed protein, a fee vitamin and mixture thereof. In some embodiments, the enhanced animal health is selected from the group consisting of: reduced or eliminated microbial infection, reduced or eliminated fungal infection, reduced or eliminated viral infection, reduced or eliminated oxidative stress, reduced or eliminated infection or death during transport, increased body weight, increased rate of weight gain, increased growth rate, reduced or eliminated birth mortality, increased or improved body score conditions, increased reproductive success, and increased gut health maturation.

The present disclosure provides a method of delivering an active agent to a subject, the method comprising: applying to the subject with a composition that comprises a minicell and an active agent taught herein. In some embodiments, the subject is an animal. In some embodiments, the active agent is selected from a nucleic acid, a peptide, a protein, an essential oil, and combinations thereof. In some embodiments, the active agent has an antimicrobial, antibacterial, antifungal or antiviral activity. In some embodiments, the active agent is a veterinary drug for animal vaccination or immunization. In some embodiments, the animal vaccination or immunization is against bacterial infections, gastric disorders, viral infections, cancer, parasite infections, non-infectious diseases, fertility and production control.

### EXAMPLES

The following examples are given for the purpose of illustrating various embodiments of the disclosure and are not meant to limit the present disclosure in any fashion. Changes therein and other uses which are encompassed within the spirit of the disclosure, as defined by the scope of the claims, will occur to those skilled in the art.

### Example 1. Production and Characterization of AgriCell (Minicell) Platform

A minimal media fermentation and a two-step centrifugation process constitute the manufacturing process for AgriCell production and purification. The entire process takes approximately 36 hours to produce and recover 20-50 grams dry mass of AgriCells per liter of fermentation broth. Pure AgriCells were concentrated in PBS using centrifugation and then frozen at −80° C. Samples were then sealed inside the LabConco FreeZone Plus 6 system and lyophilized overnight at a chamber temperature of −90° C. and pressure of 133×10⁻³ mBar.

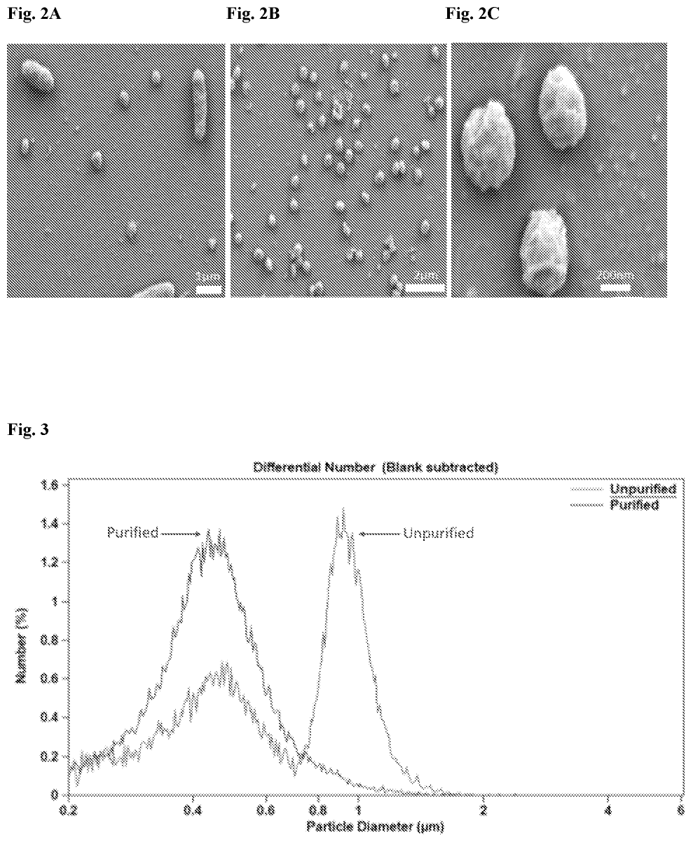

An E. coli strain was taken and designed for a fermentation process to robustly produce AgriCells. A downstream process was developed to rapidly and effectively purify the bacterial minicells from the viable, whole parental cells. FIG. 2A-2C shows scanning electron microscopy (SEM) images of the whole, rod-shaped cells (FIG. 2A) and smaller, spherical AgriCells after a fermentation and purification process (FIG. 2B-2C). All images were taken with the Zeiss Sigma VP HD field emission SEM in secondary electron imaging mode.

The efficacy of a differential centrifugation protocol developed by inventors is further demonstrated in FIG. 3, which shows a size distribution profile of AgriCell producing cell line. The profile was generated using the Multisizer 4E Coulter Counter which detects and characterizes particles using electrical zone sensing. The small, anucleate AgriCells, which purified to a degree of more than 99% purity, are then taken for next step for the encapsulation of active ingredients/agents including polynucleotides, peptides, proteins/enzymes and an essential oils.

### Example 2. Encapsulation of Essential Oils (EOs) into AgriCell Platform

(1) Selection of Essential Oils (EOs)

Three active ingredients of model EOs were selected for encapsulation and efficacy studies on AgriCell. Eugenol (99% purity, extracted from clove oil, Sigma Aldrich Lot #STBJ0145), thymol (98.5% purity, extracted from thyme oil, Sigma Aldrich Lot #SLCF3572) and pyrethrum (>50% sum of pyrethrines, extracted from chrysanthemum oil, Sigma Aldrich Lot #BCCB9487) are prepared.

Also, genistein (98% purity, isolated from glycine max soybean, Sigma Aldrich Lot #G6776), carvacrol (98% purity, produced by aromatic plants, Fisher Scientific Lot #11-101-8971), and geraniol (98% purity, isolated from palmarosa oil, Sigma Aldrich Lot #SHBL9235) are also prepared.

(2) Loading Process

Phytobiotics (e.g. eugenol, pyrethrum and thymol) were selected as model EOs for encapsulation experiments. Also, model antimicrobials (e.g. genistein or eugenol) were selected as model EOs for encapsulation experiments.

After AgriCells are purified, the concentrated AgriCell paste or dry lyophilized powder is loaded with EOs. If dry, the AgriCells are first homogenized into a finer powder through mechanical homogenization. A stock solution from each EO is prepared in ethanol at 100 mg/mL-200 mg/mL. Lyophilized AgriCells would then be suspended in a solution with the EO solution at a ratio of 1 g of dry AgriCells to 10 mL of EO solution. Once resuspended, the ethanol is allowed to evaporate overnight, leaving behind EO encapsulated AgriCells in the process. After this overnight period, encapsulated AgriCells are removed from the beaker, and the EO-encapsulated AgriCell powder is mechanically homogenized. This encapsulated product is ready for resuspension in its appropriate medium. Loading efficacy was measured and/or calculated as percentage of EO loaded into AgriCell after extraction with ethanol 100% v/v and quantification by UV-Vis spectroscopy. Eugenol and thymol were quantified at 280 nm, whereas pyrethrum was quantified at 230 nm against its respective calibration standard curve. Also, Genistein was quantified at 500 nm, whereas eugenol was quantified at 280 nm against its respective calibration standard curve.

Encapsulation of Active Ingredients/Agents of EOs into AgriCell Platform

The active ingredients/agents of model essential oils (such as eugenol, thymol, pyrethrum, and genistein) were efficiently encapsulated into AgriCell platform, via passive diffusion—concentration reduction mechanism.

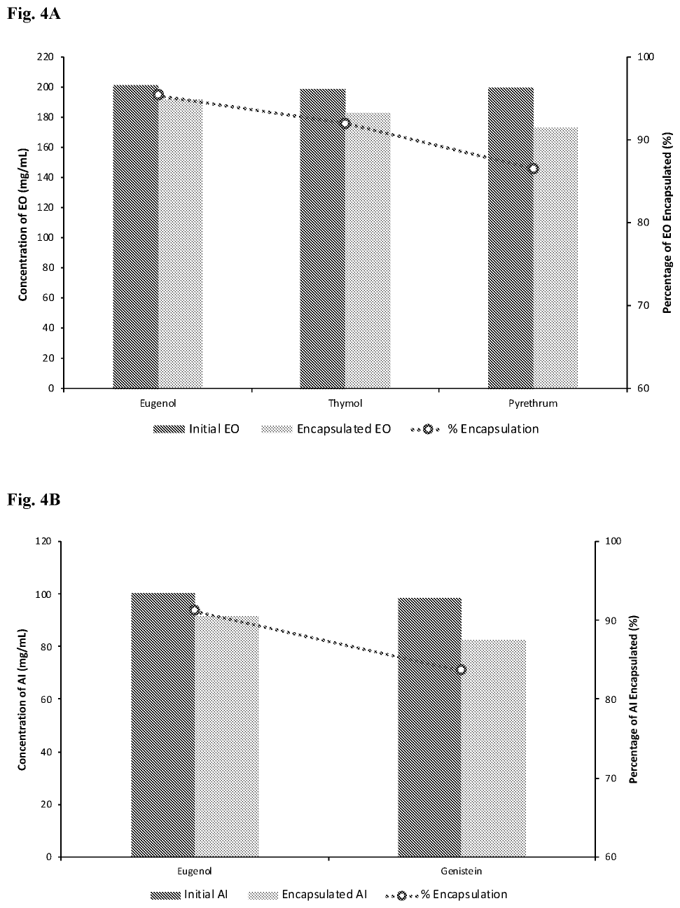

FIG. 4A shows the results for encapsulation efficacy of eugenol, thymol, and pyrethrum, and the final concentrations of EOs encapsulated into the AgriCell. Results indicate all model essential oils showed good yields for encapsulation, with eugenol showing the highest encapsulation efficacy (95.5%), followed by thymol (91.8%) and the lowest yield by pyrethrum (86.5%). All formulations showed optimal stability and were easy to handle.

FIG. 4B shows the results for encapsulation efficacy of eugenol and genistein, and the final concentrations of EOs encapsulated into the AgriCell. Results indicate all model antimicrobial AIs showed good yields for encapsulation, with eugenol showing the highest encapsulation efficacy (91.1%) and genistein showing slightly lower percentage (83.6%). All formulations showed optimal stability and were easy to handle.

### Example 3. Stability of Essential Oils (EOs) Encapsulated into AgriCell Platform

(1) Improved Chemical Stability of EOs to Changes in pH

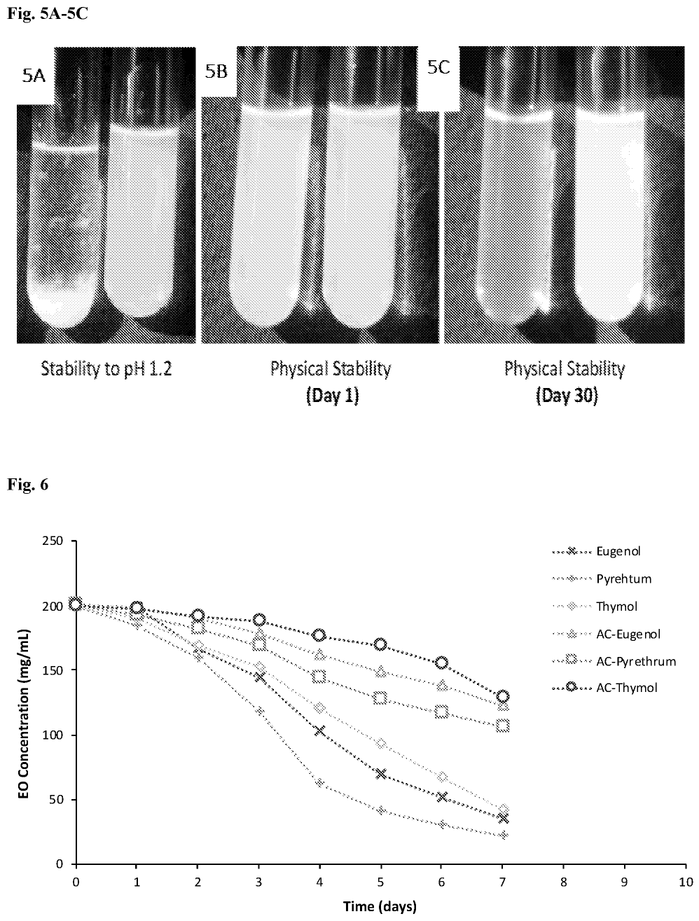

For illustrative purposes, FIG. 5A-5C shows the physical stability of the model formulation composed by eugenol encapsulated AgriCell, when compared to a standard liposomal formulation encapsulating eugenol, using soybean lecithin and cholesterol. FIG. 5A illustrates AgriCell encapsulating eugenol (right tube), which shows improved chemical stability to changes in pH, when compared to Eugenol-encapsulating liposomal formulation (left tube). AgriCell-encapsulated eugenol showed improved stability when pH was adjusted to simulate gastric conditions (pH 1.2). FIG. 5B-5C illustrates the improved physical stability of AgriCell-encapsulated eugenol (right tube) against a Eugenol-encapsulated liposomal formulation (left tube) on day 1 (FIG. 5B) and day 30 (FIG. 5C) after storage under controlled conditions (temperature 25° C., relative humidity 30% and pH 7.2). All samples were diluted 1:10 with deionized water.

Results present that AgriCell platform succeeded in stabilizing the encapsulated EO (e.g. eugenol) when compared to a standard liposomal formulation, showing better stability to changes in pH (FIG. 5A) and under controlled storage conditions (temperature 25° C., relative humidity 30% and pH 7.2; FIG. 5B-5C).

As shown in FIG. 5A, the EO-encapsulated liposomes suffered depletion flocculation processes when submitted to changes in pH 1.2 simulating gastric conditions, depicting in immediate release of encapsulated EO and poor bioavailability without desired nutritional effects. As shown in FIG. 5B-5C, the same liposomal formulation lacked long term stability and the formulation experienced significant degradation after 30 days storage under controlled conditions. Thus, results indicate that AgriCell succeeded in providing improved stability, higher bioavailability and extended shelf life for encapsulated EOs.

(2) Improved Thermal Stability of EOs

AgriCell-encapsulated EOs (200 mg/mL of EO loaded with 200 mg/mL of AgriCell) were diluted 1:10 in deionized water (total volume 1000 μL, 14 replicates for each EO) and the solutions were left at 40° C. for a total period of 7 days. One replicate of each EO per treatment was collected daily and tested for EO content. Results were reported as concentration of EO as function of time for each stability condition.

Ambient temperature crucially influences essential oil stability in several respects. Generally, chemical reactions accelerate with increasing heat due to the temperature-dependence of the reaction rate as expressed by the Arrhenius equation. Based thereon, the van't Hoff law states that a temperature rise of 10° C. approximately doubles chemical reaction rates, a relation that can be consulted to predict stability at different temperatures (Glasl, 1975). Hence, both autoxidation as well as decomposition of hydroperoxides advances with increasing temperature, even more so since heat is likely to contribute to the initial formation of free radicals (Choe and Min, 2006).

FIG. 6 shows the performance of AgriCell in preventing thermal degradation of model EOs. Results support the improvement in EO's thermal stability when encapsulated into AgriCell. The trends in FIG. 6 showed that free pyrethrum experienced the highest sensitivity to temperature raise, followed by free eugenol and lastly free thymol, reaching percentages of degradation after day 7 of 10.8%, 17.3% and 20.9%, respectively. The same trends were seen in the AgriCell encapsulated formulations, but the percentages of degradation were significantly improved in about 45%, with AgriCell-encapsulated pyrethrum, eugenol and thymol yielding to percentages of degradation of 52.5%, 61.0% and 64.4% at day 7 of the stability experiment.

(3) Improved Oxidative Stability of EOs

Ultraviolet (UV) light and visible (Vis) light are considered to accelerate autoxidation processes by triggering the hydrogen abstraction that results in the formation of alkyl radicals. Compositional changes proceeded considerably faster when illumination is involved. Especially monoterpenes have been shown to degrade rapidly under the influence of light (Turek and Stintzing, 2013). Essential oils experiences accelerated autoxidative reactions when exposed to UV or light radiation, which triggers hydrogen abstraction that results in the formation of alkyl radicals (Turek and Stintzin 2013).

AgriCell-encapsulated EOs (200 mg/mL of EO loaded with 200 mg/mL of AgriCell) were diluted 1:10 in deionized water (total volume 1000 μL, 14 replicates for each EO) and the solutions were left under UV radiation for a total period of 7 days. One replicate of each EO per treatment was collected daily and tested for EO content by UV-Vis spectroscopy as described in Example 2. Results were reported as concentration of EO as function of time for each stability condition.

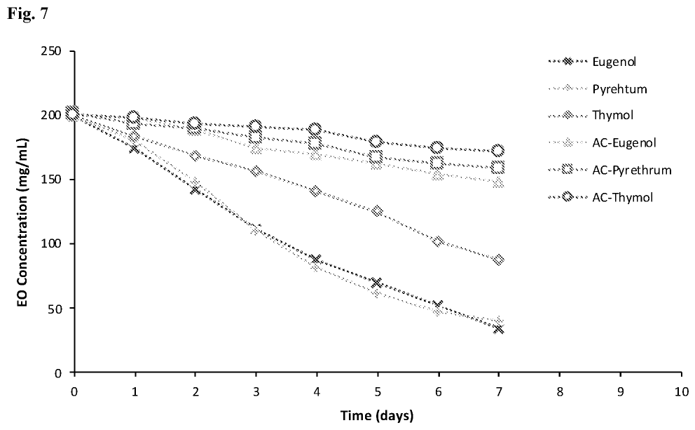

FIG. 7 shows the performance of AgriCell in preventing oxidative degradation of model EOs by influence of UV and Vis light. Results support the improvement in EO's oxidative stability when encapsulated into AgriCell. The trends in FIG. 7 show that free pyrethrum and eugenol experienced the highest oxidative rate, whereas free thymol showed lower tendency to oxidation. After 7 days of stability experiments, the oxidative processes yielded existence of 17.3%, 20.0% and 44.2% for eugenol, pyrethrum and thymol, respectively (i.e., 82.7%, 80.0%, and 55.8% of eugenol, pyrethrum and thymol, respectively were autoxidatively degraded). However, after 7 days of stability experiments, degradation rates for the model essential oils encapsulated into AgriCell were 26.0%, 20.0%, and 14.1% for eugenol, pyrethrum and thymol, respectively (i.e. 74.0%, 80.0% and 85.9% of eugenol, pyrethrum and thymol, respectively remained/not damaged). This is corresponding to an improvement of about 50% for all the encapsulated formulations over free EO formulations, supporting the protective effect of AgriCell encapsulation on autoxidation of essential oils. The results in FIG. 7 shows the effect AgriCell encapsulation on preventing autoxidative degradation of essential oils.

These results support the potential improvement in shelf-life properties for encapsulated AgriCell vaccines containing sensitive active ingredients.

### Example 4. Controlled Release of Essential Oils (EOs) Encapsulated into AgriCell Platform

(1) Controlled Release Properties of AgriCell Encapsulating Active Ingredients (AIs)

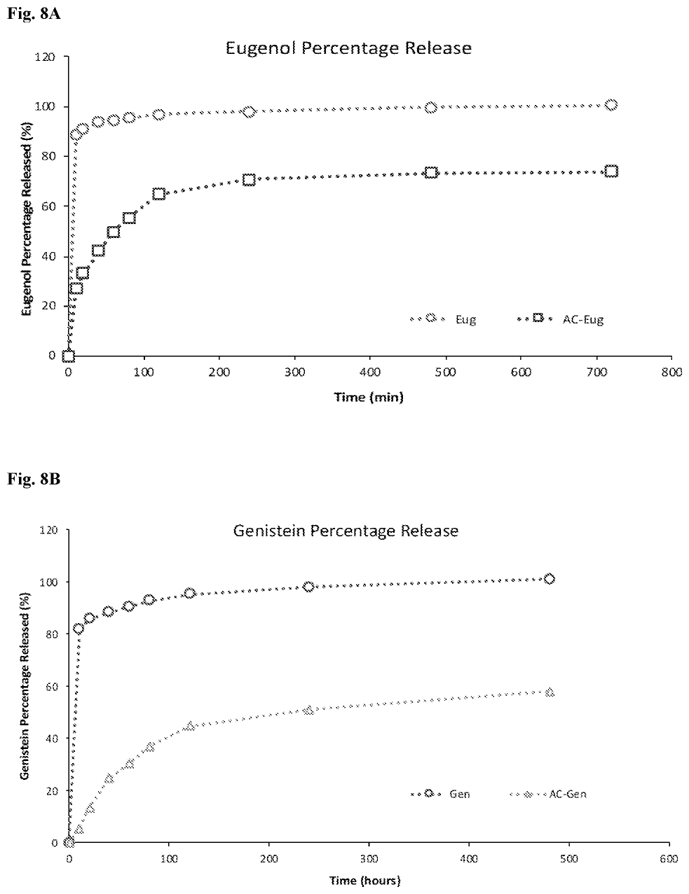

AI-loaded AgriCell formulations were prepared in PBS (1×, pH 7.4) and diluted to a known concentration in release media composed by PBS, ethanol and Tween 80 emulsifier (140:59:1 v/v/v). Samples (500 μL) were loaded into dialysis cassettes (MWCO 8-10 kDa) pre stabilized in deionized water, place into a reservoir container filled with exactly 100 mL of release media and kept under gentle stirring at room temperature. At different time intervals, an aliquot (1000 μL) of release media was removed for quantification of released AIs in ethanol (100%v/v) performed by UV-vis spectrometry as described above, a new volume of fresh release media was added to continue release experiment. AIs released from AgriCell platform were reported as percentage cumulative release over the selected timeframe, as shown in FIG. 8A-8B, corresponding to the release profiles for eugenol (FIG. 8A) and genistein (FIG. 8B), respectively.

Results indicate AgriCell platform can efficiently delay burst release stage of antimicrobial AIs (e.g. eugenol and genistein), as suggested by the significant reduction in the percentage of each AI released in the first hour of the release experiments, where all encapsulated AIs showed percentages of release lower than 40%, whereas free AIs have reached close to 90% release in the same timeframe. After completion of release studies, AgriCell encapsulated antimicrobial AIs reached a percentage release of 58.1% and 73.9%, for genistein and eugenol, respectively. In average, AgriCell encapsulation was able to improve controlled release of antimicrobial AIs in about 35% for all model AIs tested.

These values can be significantly improved by surface coating AgriCell platform with chitosan biopolymer, yielding to an additional improvement in controlled release of encapsulated AIs at least between 15 to 20%, as described below.

(2) Chitosan Coating Process to Modify AgriCell Controlled Release Properties

AgriCell-encapsulated EO (AgriCell-EO) formulations can then be further modified for its release properties. Surface coating technique via ionotropic gelation mechanism was used to generate a unique AgriCell-EO formulation, composed by an EO encapsulated by AgriCell that is coated by chitosan biopolymer (AgriCell-EO CHT).

Existing studies have shown that to optimize the characteristics and stability of carriers which can be coated by a biopolymer, by means of electrostatic interactions providing a dense polymeric shell around the carriers that will promote stabilization and prevent leaking of active ingredient to external compartments (Filipović-Grcic et al. 2007, Mengoni et al. 2017, Madrigal-Carballo et al. 2009, 2010). In this example, AgriCell platform was coated by chitosan through ionic gelation reaction due to electrostatic interactions between the negatively charged AgriCell surface and the positive charges of primary amino groups in chitosan, similar to previously described for chitosan coated liposomes. Chitosan solution, in acetic acid, was mixed with AgriCell platform, dispersed in PBS (1×, pH 7.4) and previously loaded with EOs (eugenol and thymol), under continuous stirring for 1 hour at room temperature, yielding chitosan coated AgriCell-EO that were purified by centrifugation (12,000 rpm) and stored at 4° C. until further experimentation.

(3) Controlled Release of EOs from AgriCell and AgriCell-CHT

EOs-loaded AgriCell formulations (with and without chitosan surface coating) were prepared in PBS (1×, pH 7.4) and diluted to a known concentration in release media composed by PBS, ethanol and Tween 80 emulsifier (140:59:1 v/v/v). Samples (500 μL) were loaded into dialysis cassettes (MWCO 8-10 kDa) pre stabilized in deionized water, place into a reservoir container filled with exactly 100 mL of release media and kept under gentle stirring at room temperature. At different time intervals, an aliquot (1000 μL) of release media was removed for quantification of released EOs in ethanol (100%v/v) performed by UV-vis spectrometry as described above, a new volume of fresh release media was added to continue release experiments. EOs released from AgriCell platform were observed as percentage cumulative release over the selected timeframe. Original content of EOs loaded into AgriCell and the remained content after release studies were quantified by solvent extraction with ethanol (100% v/v) directly from AgriCell.

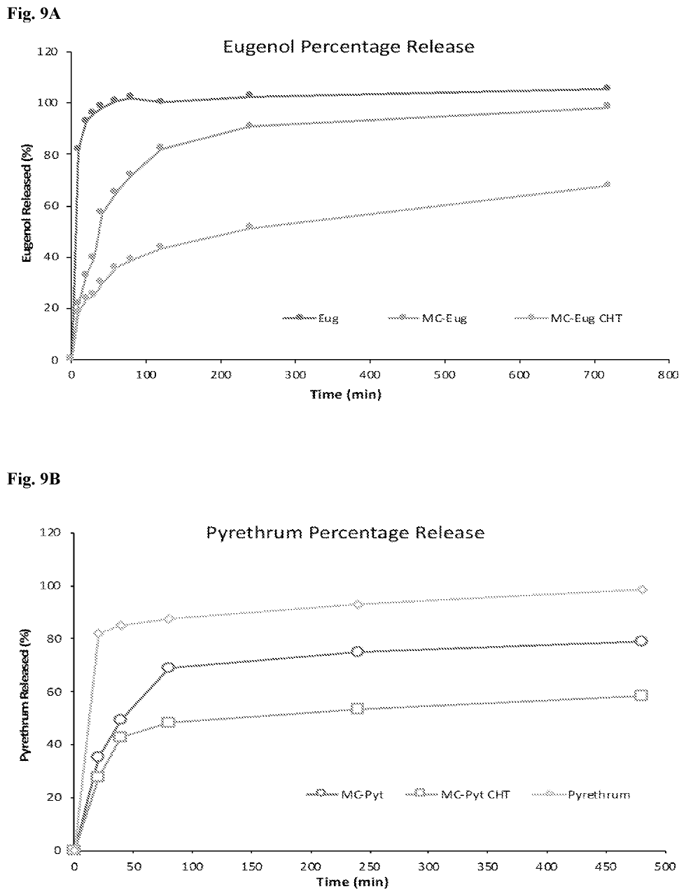

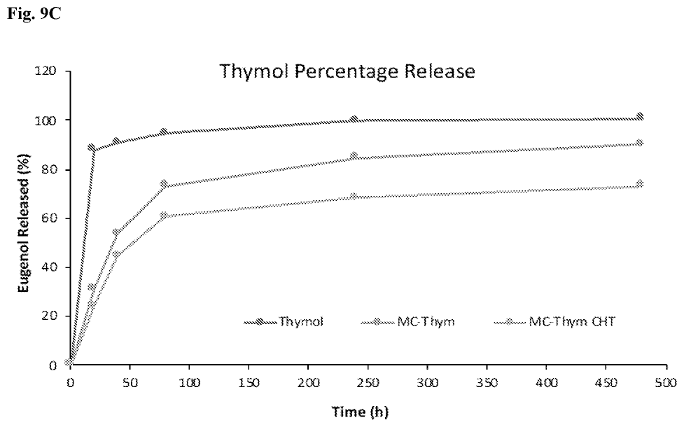

The cumulative release profile of model EOs was calculated by determining the concentration of each EO in the release medium at different times. FIG. 9A-9C shows the release profiles for eugenol (FIG. 9A), pyrethrum (FIG. 9B) and thymol (FIG. 9C) loaded into AgriCell formulations, respectively. Results indicate AgriCell platform can efficiently delay burst release stage of EOs, as suggested by the significant reduction in the percentage of each EO released in the first hour of the release experiments, where all encapsulated EOs showed percentages of release lower than 40%, whereas free EOs have reached close to 90% release in the same timeframe.

After completion of release studies, AgriCell encapsulated EOs reached a percentage release of 98.5%, 79.0% and 90.1% for eugenol, pyrethrum and thymol, respectively, as shown in FIG. 9A-9C. Similar behavior was observed for the treated AgriCell systems surface coated by chitosan biopolymer (CHT), but the CHT coated systems showing a more efficient delaying effect on release of EOs when compared to AgriCell non-coated, yielding to final percentages of release at the end of the experiment of 67.8%, 58.3% and 73.0% foe eugenol, pyrethrum and thymol, respectively. In average, chitosan coating of AgriCell was able to improve controlled release of encapsulated EOS in about 20% for all model EOs tested.

### Example 5. Bacterial Agglutination Properties of EOs Encapsulated into AgriCell

E. coli strain ATCC 8739 (which causes disease outside of the intestinal tract and its highly virulent in chickens) from a frozen stock was cultured in tryptose broth (10 g of tryptose, 2.5 g of sodium chloride, 0.5 g of dextrose, and 0.0025 g of thiamin hydrochloride in 500 mL of deionized water) at 37° C. for 48 hours. After incubation, 1 mL of E. coli cultures were taken from the surface of the broth and used to inoculate new broths that were incubated at 37° C. for 24 hours. After 24 hours, the bacterial cultures were centrifuged at 1800 g for 10 minutes to obtain bacterial pellets. The pellets were washed twice with 1x PBS+Ca²⁺/Mg²⁺ and then resuspended in 1 mL of PBS+Ca²⁺/Mg²⁺ to obtain the bacterial stock solutions. The optical density of the suspension was measure at 600 nm and bacterial cell densities (CFU/mL) were calculated using previously established bacterial growth curves for each strain of bacteria. Bacterial agglutination was measure on 950 μL of testing samples (free genistein and AC-genistein) were diluted to the tested concentrations in 1x DPBS+Ca²⁺/Mg²⁺ were added to a cuvette and mixed with 50 μL of the bacteria stock solution of 1.0×10¹⁰ CFU/mL. The final concentration of each cuvette was 300 μg/mL of genistein equivalent and 5.0×10⁸ CFU/mL in a total volume of 1 mL. The cuvettes were mixed via trituration and placed on an UV-Visible spectrophotometer equipped with an eight-position cuvette holder. The percent transmittance of light at 450 nm was measured every 1 min for 240 min. The interaction of genistein and AC-genistein (genistein encapsulated into AC) with the bacteria resulted in an agglutination and precipitation of the bacteria from solution. After 240 min, the area under the curve was calculated from the generated transmittance calibration curves.

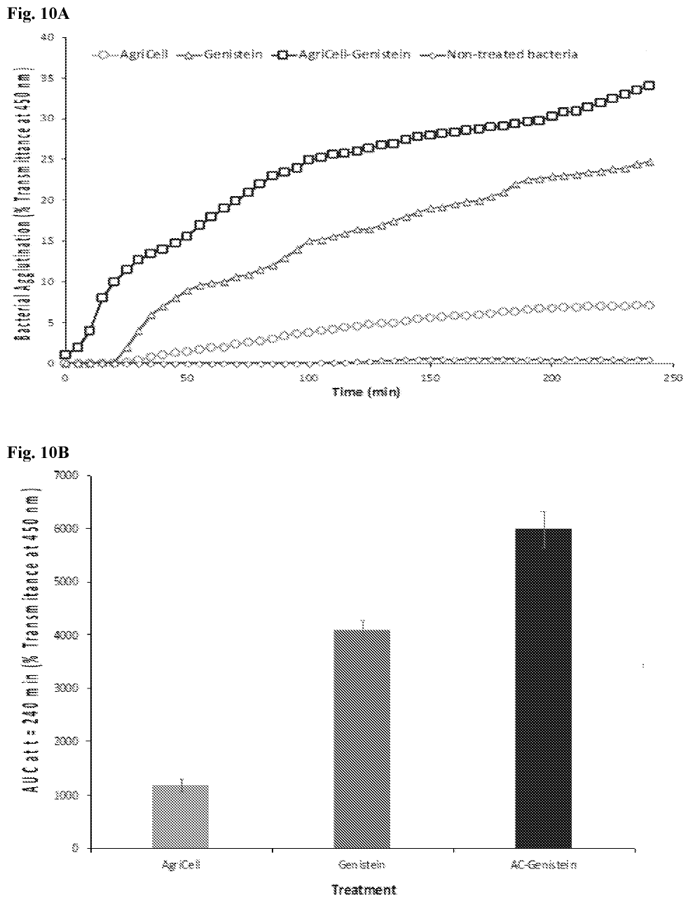

FIG. 10A-10B shows the increase in transmittance of light (450 nm) that results from the interaction of genistein and AC-genistein with E. coli, causing agglutination and precipitation from suspension.

The results indicate that at a fixed concentration of 300 μg/mL of AC-genistein, significantly increased E. coli agglutination compared to genistein alone. FIG. 10A describes the agglutination experiment for free genistein and AC-genistein. Two different phases in the agglutination curve for genistein were observed. The first was a lag phase that last for about 25 minutes, and the second was an exponential phase from that continues for the 240 minutes run of the whole experiment. The lag phase is described as a period where genistein starts to interact with E. coli's outer membrane forming a pseudo-colloidal suspension without appreciable precipitation. After 25 minutes, precipitation due to direct genistein interactions with E. coli begins to accelerate exponentially for the rest of the experiment. In contrast, agglutination curve for the AC-genistein system showed a faster agglutination process, with no appreciable lag phase and one exponential phase starting as soon as the AC-genistein particles are in contact with E. coli, which was extended over the length of the experiment. This exponential phase could be the product of a synergistic effect between genistein and the AgriCell triggering a direct interaction between the AC-genistein and key components at E. coli's membrane surface, thus causing a faster agglutination and/or precipitation. In addition, results indicate encapsulation of genistein into AgriCell successfully increased its bacterial agglutination properties in more than 30%, after 240 min incubation with pathogenic E. coli strain 8739 (FIG. 10B).

### Example 6. Bacterial Inhibition Properties of EOs Encapsulated into AgriCell

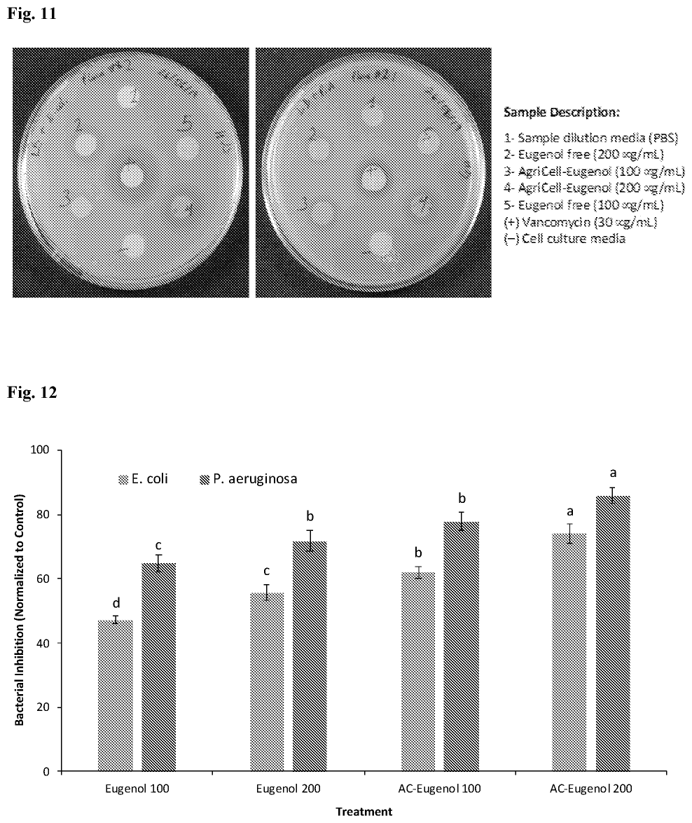

In this example, the zone of inhibition test, also called a Kirby-Bauer Test, was conducted to measure antibiotic resistance of eugenol encapsulated into AgriCell. Approximately 1×10⁸ cells from a selected bacterial strain (Escherichia coli ATCC 25922 and Pseudomonas aeruginosa ATCC 15442) were spread over an agar plate using a sterile swab, then incubated in the presence of the antimicrobial agent (adsorbed over a paper disc). If the bacterial strain is susceptible to the antimicrobial agent, then a zone of inhibition appears on the agar plate, whereas if the selected bacterial strain is resistant to the antimicrobial agent, then no zone is evident. FIG. 11 shows the results for antibacterial susceptibility testing on free eugenol and AgriCell-eugenol at two final dilution doses for eugenol (100 and 200 μg/mL).

Results indicates that encapsulation of eugenol into AgriCell (AC-eugenol) significantly improved its antimicrobial inhibition effect against E. coli and P. aeruginosa, quantitative analysis of the plate assay is shown in FIG. 12, indicating encapsulation of eugenol into AgriCell yielded to increased bacterial inhibition when compared to free eugenol that showed to be statistically significant even for the lower doses of eugenol treated (100 μg/mL). Compared to the positive control (Vancomycin 30 μg/mL), AC-eugenol at 200 μg/mL effectively inhibited about to 90% and 80% of E. coli and P. aeruginosa growth in vitro, respectively, whereas free eugenol at 200 μg/mL only reached less than 80% and 60% growth inhibition for the respective bacterial strains. Additionally, AC-eugenol at the significantly lower dose of 100 μg/mL reached percentages of inhibition similar to those of free eugenol at 200 μg/mL, inhibiting bacterial growth in more than 80% and 60% for E. coli and P. aeruginosa, respectively. Thus, encapsulation of eugenol into AgriCell could promote a synergistic effect between eugenol and the AgriCell triggering a direct interaction with the bacterial membrane surface that increases eugenol's growth inhibition effect.

AgriCell was successfully applied for encapsulation of model antimicrobial active ingredients/agents (e.g. genistein and eugenol), widely used in animal health vaccination.

Examples 3-6, present that encapsulation into AgriCell improved physical stability of active ingredients/agents (AIs) including essential oils, promoted controlled release properties and enhanced antibacterial properties of encapsulated AIs via a synergistic effect that increased efficacy in vitro in at least about 20-30% for both genistein and eugenol.

### Example 7. Encapsulation of Model Protein Antigen Agents in AgriCell Platform for Animal Health Vaccination

In this example, inventors tested effects of hen egg-white lysozyme (HEL) encapsulated into AgriCell platform (AgriCell-HEL) on macrophage antigen presentation of HEL, by measuring the production of interlukin-2 (IL-2) by a T-cell hybridoma line cocultured with murine peritoneal macrophages in the presence of HEL. Inventors also studied the effect of AgriCell-HEL on macrophage uptake of the fluorescently labeled protein by direct confocal microscopy observation.

(1) Loading of Hen Egg-White Lysozyme Encapsulated into AgriCell

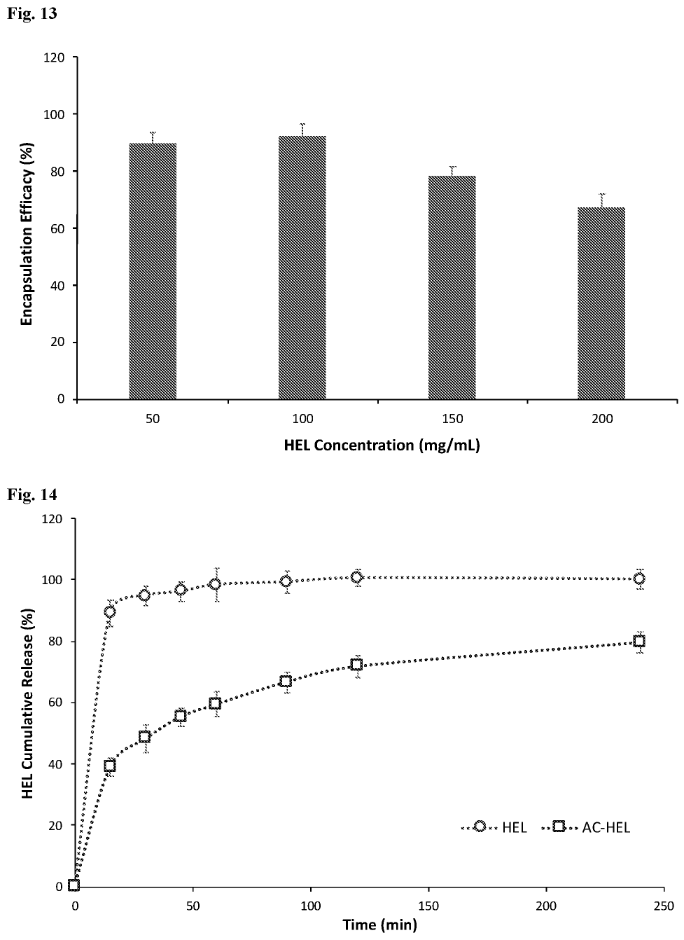

Hen egg-white protein (HEL 95% protein, molecular weight, 14.7 kDa) was obtained from Sigma-Aldrich (St. Louis, Mo., USA) and used without further purification. AgriCell dispersion in PBS was mixed with an HEL at concentrations ranging 50 to 200 mg/mL. Loaded cells were quantified for its HEL loading efficacy by the Lowry protein assay. Results are summarized in FIG. 13, indicating AgriCell (100 mg/mL) efficiently encapsulated about 92.3% of HEL at a concentration of 100 mg/mL in weight ratio 1:1. The encapsulation efficiency of AgriCell decreases as the concentration of HEL increased to 150 and 200 mg/mL, showing encapsulation efficiencies of only 78.1% and 67.4%, respectively.

### Example 8. Controlled Release of Hen Egg-White Lysozyme (HEL) Encapsulated into AgriCell Platform

HEL release from AgriCell platform was determined in PBS (pH 7.3). The loaded AgriCell-HEL were resuspended in PBS (pH 7.3) to make a 10 mg/mL HEL dose. Samples were incubated at 37° C. under mild shaking. After 15, 30, 45, 60, 90, 120, 180, and 240 minutes, the tubes were given a spin-off and samples of 250 μL of the supernatant were taken. These samples were replaced by PBS (pH 7.3). The non-encapsulated HEL in PBS was determined with the Lowry protein assay and AgriCell-encapsulated HEL was determined by arithmetic differential calculation. Release profiles for HEL shown in FIG. 14 indicate that AgriCell successfully promoted a controlled release profile for HEL, reducing the initial burst stage at 15 minutes from 89.2% released for free HEL, to as low as 38.9% released for HEL encapsulated into AgriCell. After release experiments were concluded at 240 minutes, free HEL reached 100.1% released, whereas AgriCell-HEL has only released 79.6% of the originally encapsulated HEL.

### Example 9. Effects of Model Protein Antigen Agents in AgriCell Platform on Immune Response Activation of HEL

Mouse peritoneal macrophages were resuspended in media and plated in 24-well cell-culture plates at a concentration of 1×10⁸ cells/well. After 3 hours, the macrophages were attached and washed with DMEM media until cells other than macrophages were removed. Supplemented media (DMEM, 10% FBS, 2 mM of L-alanyl-L-glutamine, 100 U/mL of media penicillin and 100 U/mL of media streptomycin) was added and the macrophages were incubated overnight at 37° C. and 5% CO₂. The 3A9 T-cell hybridomas were maintained in DMEM supplemented with 10% FBS, 50 μg of gentamycin/mL media, 2 mM of L-alanyl-L-glutamine, 10 mM of HEPES buffer, 55 μM of 2-mercaptoethanol, and 1× non-essential amino acids. For experiments, media was removed from the macrophages and rinsed once with PBS. Media was then supplemented with free HEL (10 mg/mL) or AgriCell-HEL (10 mg/mL), added to the macrophages and incubated for 0.25 to 8.0 hours. The media was removed, and cells washed once with PBS. The T-cell hybridomas (concentration equals 2× the number of macrophages) in 3A9 media (without phenol red) were added to the macrophages and incubated for 24 hours. Media was removed, placed in microfuge tubes, centrifuged for 4 minutes at 15,000×g, and IL-2 was measured by an ELISA kit (555148, BD Biosciences). Data were expressed as pg of IL-2/mL media. Results shown in FIG. 15 indicate that, after 4 hours of preincubation, only trace amounts of IL-2 were detected in the cocultures treated with HEL alone, whereas cocultures treated with AgriCell-HEL had an important production of IL-2 at 2 hours preincubation and the AgriCell-HEL reached maximum IL-2 expression at timepoint 6 hours. In the absence of AgriCell, IL-2 expression increased with increasing time of incubation, but after completion of the experiment (that is 10 hours preincubation) HEL has not reached its maximum IL-2 expression. AgriCell free treatment did not seemed to affect IL-2 expression, suggesting AgriCell-HEL may increase the uptake of HEL and subsequent expression of IL-2 by the T-cell hybridomas. Alternatively, AgriCell-HEL may decrease the rate of HEL proteolysis in the macrophage, allowing more antigenic peptide to be presented to the T-cell hybridoma.

Furthermore, mouse peritoneal macrophages were cultured in glass-bottomed culture plates and treated with HEL and AgriCell-HEL. Endocytosis and subsequent proteolysis of HEL in the endosomes were studied by fluorescent microscopy with fluorescently labeled HEL (quenched BODIPY dye conjugate A-20181, Molecular Probes). HEL was labeled according to a protocol provided by kit and subsequently loaded into AgriCell, according to the methodology described above. Macrophages were incubated with each treatment for 0.25 to 8.00 hours and imaged by fluorescent microscopy using 450-490 nm excitation and 510-565 nm emission filters. Images were captured after 30 min incubation. The microscope was fitted with a chamber to maintain the cells at 37° C. and 5% CO₂.

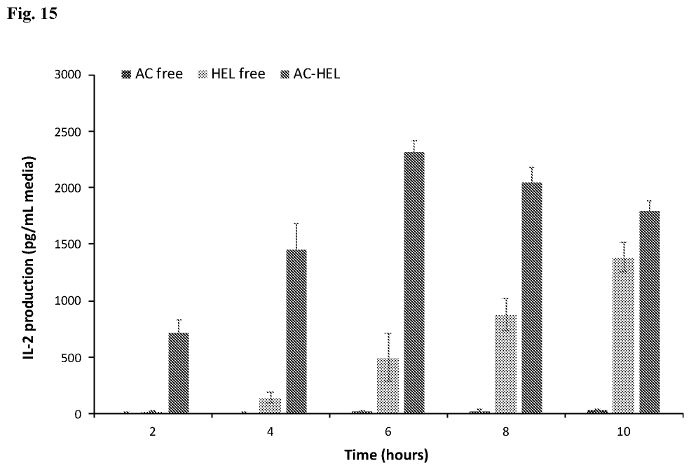

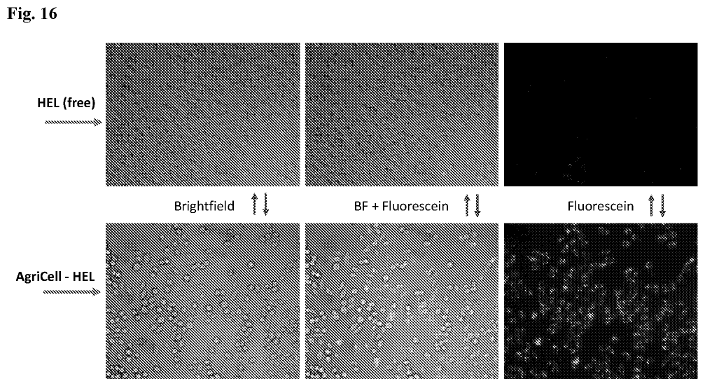

Fluorescently labeled HEL loaded into AgriCell was incubated with mouse peritoneal macrophages, and uptake of free HEL and AgriCell-HEL was followed by fluorescent microscopy, as shown in FIG. 16. After 30 minutes of incubation, macrophages treated with AgriCell-HEL clearly contained more fluorescent endosomes than the macrophages treated with free HEL. Since uptake by macrophage cells is the first step in vaccination, the results suggest that AgriCell platform is a promising immunoadjuvant for vaccine delivery systems.

In vitro studies have showed that AgriCell-HEL can increase macrophages uptake and also modulate antigen presentation of model protein HEL. Attenuation of gut macrophage activation by antigenic proteins loaded into AgriCell may be beneficial in the treatment and prevention of gut-related infectious and inflammatory diseases, promoting AgriCell encapsulation as a great immunoadjuvant for oral vaccination.

The broad spectrum of possible applications of AgriCell platform technology allow for a safe and sophisticated product for targeted delivery of active ingredients/agents (including peptides, proteins and/or enzymes) as a vaccine as well as carrier of immobilized enzymes for applications in animal health.

### Example 10. Characterization, Purification, and Stability of Minicells Encapsulated-dsRNAs

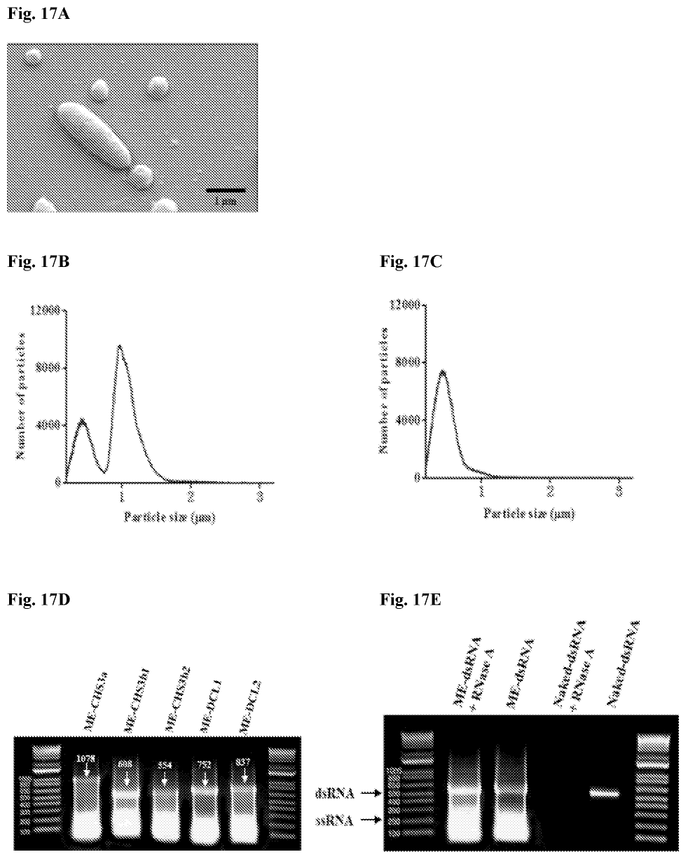

Minicells are bacterially-derived achromosomal microparticles, generally produced as a result of aberrant cell divisions. Similar to the parental cells, minicells contain membranes, ribosomes, RNA and proteins; but unlike normal cells, they cannot divide or grow (Farley et al 2016). This unique feature has led to the development of minicells as human therapeutic delivery vesicles for drugs, vaccines, and siRNAs (MacDiarmid et al 2007; Carleton et al 2013; MacDiarmid et al 2009). Deletions of the E. coli cell cycle-related genes minCDE produce a large number of intact and stable minicells (MacDiarmid et al 2007; Hale et al 1983). Inventors generated an E. coli mutant (minCDE and rnc) that produces a large number of minicells with compromised RNase-III activity (FIG. 17A-17C), thus allowing the expression of dsRNAs (Takiff et al 1989). Inventors transformed this E. coli mutant with DNA constructs to express dsRNAs targeting B. cinerea genes that are essential for pathogenicity. The first group of genes inventors used are cell-wall integrity-related genes, including two isoforms of chitin synthase class III; Chs3a (ME-CHS3a) and Chs3b (ME-CHS3b1 and ME-CHS3b2). Chitin is the rigid carbohydrate polymer that constitutes the cell wall of fungi. In B. cinerea, seven different classes of the chitin synthase genes have been identified, with duplications present in class III (Chs3a and Chs3b). Deletions of Chs3a significantly reduce the virulence and radial growth of B. cinerea (Soulie et al 2006). The second group of genes inventors targeted are the B. cinereal RNAi-machinery related genes, DCL1 (ME-DCL1) and DCL2 (ME-DCL2). DCL1 and DCL2 are involved in the synthesis of fungal siRNA-effectors to suppress immunity and facilitate gray mold disease progression.

The transformed E. coli mutant was cultured in batch phase, induced for dsRNA expression and processed via differential centrifugation, to recover purified minicells at the level of purity depicted in FIG. 17C, a single peak representing the minicells. The dsRNA from the pure minicell solution was extracted and size-verified (FIG. 17D). The ability of minicells to protect dsRNAs from RNase A degradation was confirmed in vitro (FIG. 17E). Compared to naked-dsRNAs, minicells were resistant to RNase A treatment and yielded an intact dsRNA band on the agarose gel (FIG. 17E). ME-dsRNA remained unaffected by RNase A treatment, with both treated and untreated conditions producing a band of the same relative intensity, visualized with native agarose gel electrophoresis. These results indicate that the RNase cannot access dsRNA that is minicell-encapsulated. The naked-dsRNA was degraded beyond the limit of detection given the same RNase A treatment, while the non-treated naked-dsRNA band was visualized using the same method indicating that the RNase A was responsible for the degradation.

### Example 11. Effects of the Minicells Encapsulated dsRNAs (ME-dsRNAs)

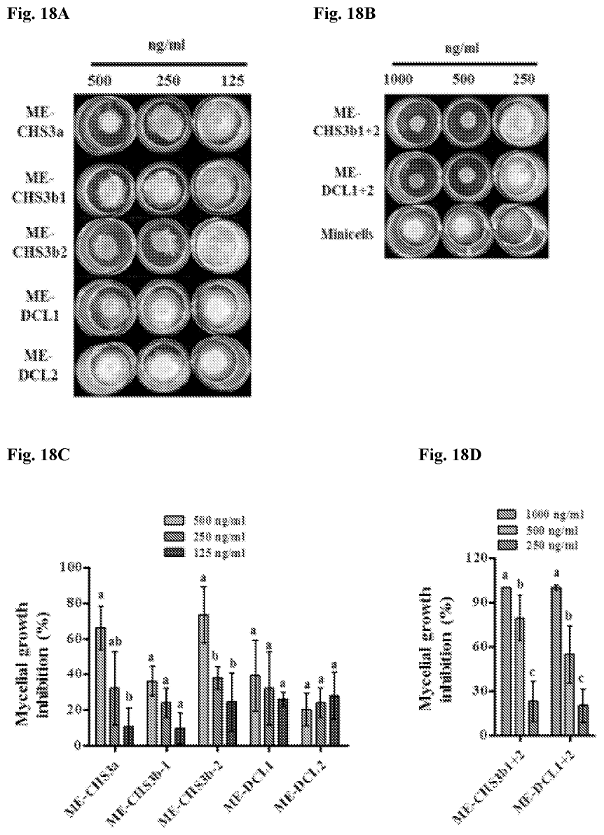

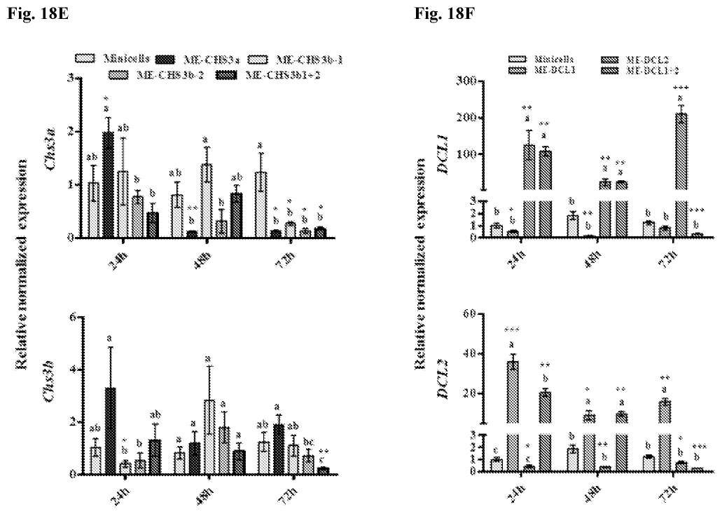

The potential of ME-dsRNAs to function as an effective biofungicide was examined in vitro by exposing actively growing fungal mycelia of B. cinerea to ME-dsRNAs. As shown in FIG. 18A and 18C, both 500 ng/mL of ME-CHS3a and ME-CHS3b2 showed significant antifungal activity, and the combination of the two constructs targeting Chs3b (CHS3b1+2 at 1000 ng/mL) completely inhibited the mycelial growth FIGS. 18B and 18D). To further validate these findings at the molecular level, the relative expression of target genes was evaluated by qRT-PCR, using B. cinerea β-actin and Tubulin genes as housekeeping genes and an empty-minicell treatment as a negative control. Treatments by ME-CHS3a and the combination of ME-CHS3b1+2 significantly reduced the relative transcript level of Chs3a and Chs3b in fungal mycelia, respectively, at 72 hours post treatment (hpt). Additionally, the reduction in Chs3a observed in response to ME-CHS3b2 and ME-CHS3b1+2 at 72 hpt (FIG. 18C-18D), could be explained by the high degree of similarities (60.5%) between Chs3a and Chs3b gene homologs (Choquer et al 2004). Interestingly, it has previously been reported that Chs3b transcripts were not detected in the mycelium of B. cinerea using the semi-quantitative RT-PCR approach (Choquer et al 2003). Likewise, inventors observed significant reduction in Chs3b transcript levels in responses to ME-CHS3b1+2 treatment, however, the level of the Chs3b transcripts was generally low as indicated by the high Cq values (i.e. 35 out of 40 PCR cycles). These datasets together suggest that ME-dsRNA targeting the Chs3b gene of B. cinerea suppressed both Chs3a and Chs3b, and the simultaneous suppression of both genes of chitin synthase class III might be crucial for the complete inhibition of B. cinereal mycelial growth.

Furthermore, the combined application of ME-dsRNA targeting DCL11 and DCL2 at 1000 ng/mL inhibited fungal growth at 72 hpt (FIGS. 18B and 18D). However, the treatment of ME-dsRNA individually targeting DCL1 (ME-DCL1) or DCL2 (ME-DCL2) did not lead to any significant mycelial growth inhibition (FIGS. 18A and 18C). These results are in agreement with what has previously been reported by Wang et al., 2017, where mutations in DCL1 or DCL2 did not compromise the virulence or growth of B. cinerea, whereas the fungus double mutant dcl1+2 showed reduced virulence. Results further support their finding that silencing of both DCL1 and DCL2 is required to inhibit B. cinereal mycelial growth. In contrast, when naked-dsRNAs targeting DCL1 and DCL2 were used, no significant mycelial growth inhibition was observed. In the example, it is presented that a single ME-dsRNA application (1000 ng/mL) was significantly effective compared to empty-minicells for as long as 72 hpt. This remarkable efficacy of ME-dsRNA could be explained, at least partially, by the ability of minicells to protect dsRNAs from enzymatic degradation as demonstrated above (FIG. 17E). The robust efficacy of ME-DCL1 and ME-DCL2 was also confirmed at the molecular level using qRT-PCR. (FIG. 18E-18F).

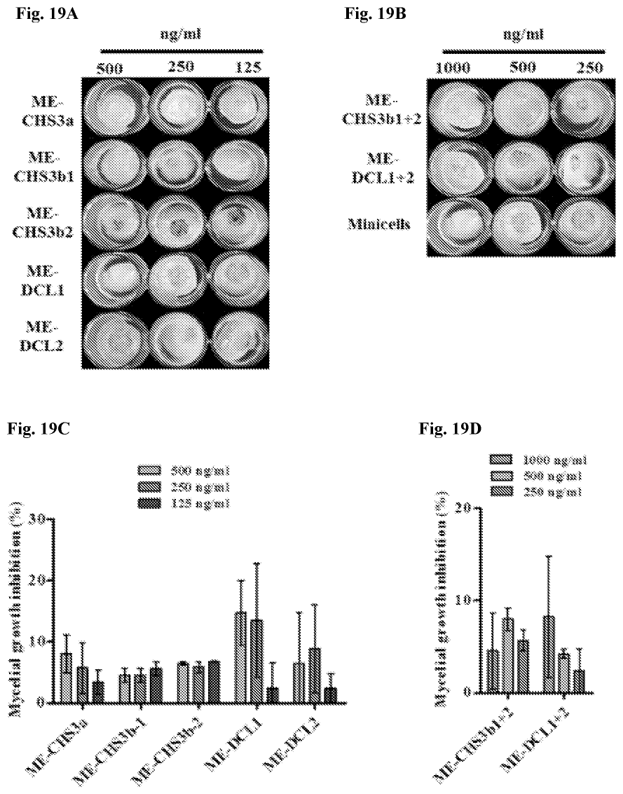

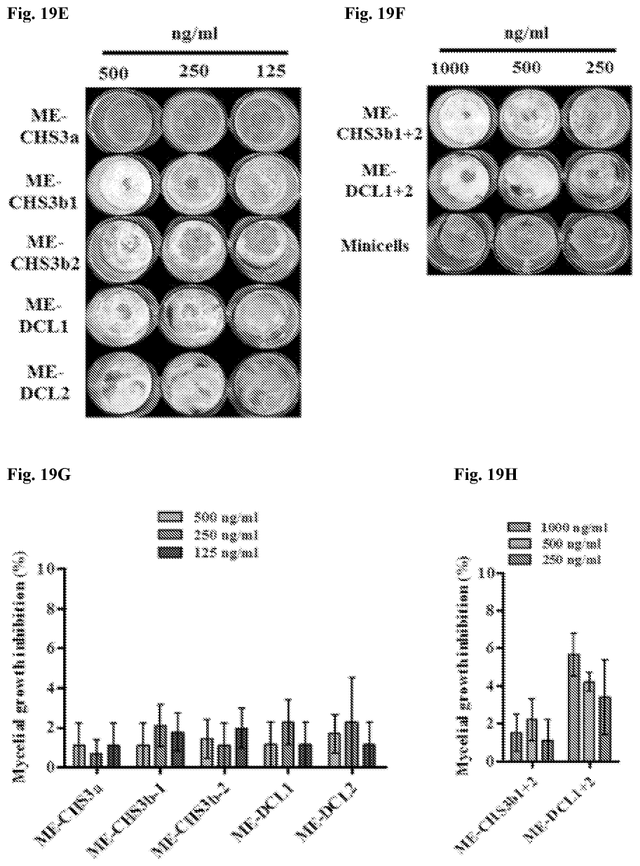

In this example, it is shown that a significant reduction in DCL1 and DCL2 transcript levels when the respective minicells were used, but inventors also observed a potential compensatory relationship between DCL1 and DCL2 expression. The downregulation of DCL1 transcripts, via ME-DCL1 treatment, was paralleled with an upregulation of DCL2 transcripts, and vice versa. The potential compensatory relationship between DCL1 and DCL2 genes and its implications in dsRNA-based management of B. cinerea is further examined. The combination of ME-DCL1+2 treatment, resulted in a spike in expression of both genes at the 24 hpt, followed by a gradual, yet significant, decline in transcript levels of both genes at 48 and 72 hpt (FIG. 18E-18F). This finding is consistent with the results in FIG. 18B and FIG. 18D where the combined, but not the single application, ME-DCL1 and 2 led to a complete inhibition of the mycelial growth of B. cinerea. The target-specific inhibition by ME-dsRNAs was also confirmed in vitro, by experimental results showing that ME-dsRNAs for B. cinerea failed to inhibit the mycelial growth of two other fruit-rot fungal pathogens, Alternaria alternata (FIG. 19A-19D), and Penicillium expansium (FIG. 19E-19H), which represents a major advantage of the ME-dsRNA as a promising class of biofungicides. These results indicates that the ME-dsRNAs designed for targeting B. cinerea genes are highly specific to its target, which can be beneficial for targeted therapeutics and vaccination.

### Example 12. Effects of the Minicells Encapsulated dsRNAs (ME-dsRNAs) for Animal Vaccination

The aforementioned minicells capable of encapsulating and delivering dsRNAs, as described in Example 10, are being used for testing encapsulation and delivery of dsRNAs to treat, cure, or prevent (i) immune diseases caused by virus taught in the present disclosure, (ii) cancers in animals, or (iii) fungal infections caused by fungal pathogens taught in the present disclosure. In this example, dsRNAs of interest are being designed for RNAi-based therapy to silence critical genes of target virus, cancer-related genes, or genes of invading pathogens or pests that are critical for virulence and disease progression.

The potential of ME-dsRNAs to function as an effective animal vaccine will be examined in vitro by exposing actively growing fungi to ME-dsRNAs. The tame gene expression in the fungi treated with ME-dsRNAs will be observed. The fungal growth inhibition assay will be performed to study effects of ME-dsRNA targeting genes in the fungi of interest. Also, the target-specific inhibition by ME-dsRNAs will be tested as a promising class of targeted therapeutics and vaccination.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### REFERENCES

AgroSpheres, Inc. Compositions and methods for pesticide degradation, PCT/2017/027048 (WO2017/180650)
AgroSpheres, Inc. Compositions and methods for enzyme immobilization, PCT/US2018/030328 (WO2018/201160)
AgroSpheres, Inc. Compositions and methods for the encapsulation and scalable delivery of agrochemicals, PCT/US2018/030329 (WO2018/201161)
AgroSpheres, Inc., Compositions and methods for scalable production and delivery of biologicals, PCT/US2018/052690 (WO2019/060903)
Hajam, I. A.; Dar, P. A.; Won, G.; Lee, J. H. Bacterial ghosts as adjuvants: mechanisms and potential. Vet. Res., 2017, 48, 37.
Bergmann-Leimer, E. S.; Leitner, W. W. Adjuvants in the driver's seat: how magnitude, type, fine specificity and longevity of immune responses are driven by distinct classes of immune potentiators. Vaccines, 2014, 2, 252-296.
Brun, A.; Barcena, J.; Blanco, E.; Borrego, B.; Dory, D.; Escribano, J. M.; Le Gall-Recule, G.; Ortego, J.; Dixon, L. K. Current strategies for subunit and genetic viral veterinary vaccine development. Virus Res., 2011, 157, 1-12.
Ebensen, T.; Paukner, S.; Link, C.; Kudela, P.; de Domenico, C.; Lubitz, W.; Guzman C. A. Bacterial ghosts are an efficient delivery system for DNA vaccines. J. Immunol., 2004, 172, 6858-6865.
Stevanovic Z. N.; Bosnjak-Neumuuller, J.; Pajic-Lijakovic, I.; Raj, J.; Vasiljevi, M. Essential Oils as Feed Additives—Future Perspectives. Molecules, 2018, 23, 1717.
Martin, G. B.; Ferasyi, T. R. Clean, Green, Ethical (CGE) Management: What Research Do We Really Need? Int. J. Trop. Vet. Biomed. Res., 2016, 1-8.
Mengoni, Y.; Adrian, M.; Pereira, S.; Santos-Carballal, B.; Kaiser, M.; Goycoolea, F. M. A chitosan—based liposome formulation enhances the in vitro wound healing efficacy of substance P neuropeptide. Pharmaceutics, 2017, 56-61.
Filipovic-Grcic, J.; Skalko-Basnet, N.; Jalsenjak I. Mucoadhesive chitosan-coated liposomes: Characteristics and stability. J. Microencap., 2007, 18, 3-12.
Madrigal-Carballo, S.; Rodriguez, G.; Sibaja, M.; Vila, A. O.; Reed, J. D.; Molina, F. Chitosomes loaded with cranberry proanthocyanidins attenuate the bacterial lipopolysaccharide induced expression of iNOS and COX-2 in Raw 264.7 macrophages. J. Liposome Res., 2009, 19, 89-196.
Madrigal-Carballo, S.; Vila, A. O.; Sibaja, M.; Reed, J. D.; Molina, M. In vitro uptake of lysozyme-loaded liposomes coated with chitosan biopolymer as model immunoadjuvants. J. Liposome Res., 2010, 20, 1-8.

Turek, C.; Stintzin, F. C. Stability of Essential Oils: A Review. Com. Rev. Food Sci. Food Safety, 2013, 12, 40-53.
Farley, M. M., Hu, B., Margolin, W. & Liu, J. Minicells, back in fashion. J. Bacterial. 198, 1186-1195 (2016).
MacDiarmid J. A. et al. Bacterially derived 400 nm particles for encapsulation and cancer cell targeting of chemotherapeutics. Cancer Cell 11, 431-445 (2007).
Carleton, H. A, Lara-Tejero, M., Liu, X. & Galan, J. E. Engineering the type III secretion system in non-replicating bacterial minicells for antigen delivery. Nat. Commun. 4, 1590 (2013).
MacDiarmid, J. A. et al. Sequential treatment of drug-resistant tumors with targeted minicells containing siRNA or a cytotoxic drug. Nature Biotech. 27, 643-651 (2009).
Hale T. L., Sansonetti, P. J., Schad, P. A., Austin, S. & Formal, S. B. Characterization of virulence plasmids and plasmid-associated outer membrane proteins in Shigella flexneri, Shigella sonnei, and Escherichia coli. Infect. Immun. 40, 340-350 (1983).
Takiff, H. E., Chen, S. & D. L. Court. Genetic analysis of the rnc operon of Escherichia coli. J. Bacteriol. 171, 2581-2590 (1989).
Soulie, M. C. et al. Botrytis cinerea virulence is drastically reduced after disruption of chitin synthase class III gene (Bcchs3a). Cell Microbiol 8, 1310-1321 (2006).
Choquer, M., Boccara, M., Gonc, alves, I. R., Soulie, M. & Vidal-Cros, A. Survey of the Botrytis cinerea chitin synthase multigenic family through the analysis of six euascomycetes genomes. Eur. J. Biochem. 271, 2153-2164 (2004).
Choquer, M., Boccara, M., & Vidal-Cros, A. A semi-quantitative RT-PCR method to readily compare expression levels into Botrytis cinerea multigenic families. Curr. Genet. 43, 303-309 (2003).
Wang, M., Thomas, N. & Jin, H. Cross-kingdom RNA trafficking and environmental RNAi for powerful innovative pre- and post-harvest plant protection. Curr. Opin. Plant Biol. 38, 133-141 (2017).
Glasl H. 1975. Uber die Haltbarkeit von Terpenoiden in Extrakten and Losungen mit unterschiedlichem Alkoholgehalt. Arch Pharm 308:88-93
Choe E, Min D B. 2006. Mechanisms and factors for edible oil oxidation. Compr Rev Food Sci Food Saf 5:169-86.

### NUMBERED EMBODIMENTS OF THE DISCLOSURE

Notwithstanding the appended claims, the disclosure sets forth the following numbered embodiments:

### Composition Comprising a Minicell and an Active Agent

1. A composition comprising: a minicell and an active agent,
   wherein the active agent is encapsulated by the minicell, and
   wherein the minicell and the active agent are present in a weight-to-weight ratio of about 5:1 to about 1:5 in the composition.
2. The composition as in any one of the preceding clauses, wherein the minicell and the active agent are present in a weight-to-weight ratio of about 1:1.
3. The composition as in any one of the preceding clauses, wherein the minicell is derived from a bacterial cell.
4. The composition as in any one of the preceding clauses, wherein the minicell is less than or equal to 1 μm in diameter.
5. The composition as in any one of the preceding clauses, wherein the active agent is a biologically active agent.
6. The composition as in any one of the preceding clauses, wherein the biologically active agent is selected from a nucleic acid, a peptide, a protein, an essential oil, and combinations thereof.
7. The composition as in any one of the preceding clauses, wherein the nucleic acid is selected from the group consisting of an antisense nucleic acid, a double-stranded RNA (dsRNA), a short-hairpin RNA (shRNA), a small-interfering RNA (siRNA), a microRNA (miRNA), a ribozyme, an aptamer, and combination thereof.
8. The composition as in any one of the preceding clauses, wherein the essential oil comprises geraniol, eugenol, genistein, carvacrol, thymol, pyrethrum or carvacrol.
9. The composition as in any one of the preceding clauses, wherein the essential oil comprises geraniol.
10. The composition as in any one of the preceding clauses, wherein the essential oil comprises eugenol.
11. The composition as in any one of the preceding clauses, wherein the essential oil comprises genistein.
12. The composition as in any one of the preceding clauses, wherein the essential oil comprises thymol.
13. The composition as in any one of the preceding clauses, wherein the essential oil comprises pyrethrum.
14. The composition as in any one of the preceding clauses, wherein at least about 10% of the active agent is encapsulated by the minicell.
15. The composition as in any one of the preceding clauses, wherein the minicell stabilizes the active agent in an acidic condition, wherein the acidic condition is less than pH 7.
16. The composition as in any one of the preceding clauses, wherein the minicell encapsulating the active agent is preserved from depletion flocculation when a pH is adjusted to an extremely acidic condition.
17. The composition as in any one of the preceding clauses, wherein the acidic condition is as low as pH 1.
18. The composition as in any one of the preceding clauses, wherein the minicell stabilizes the active agent at least 30 days at room temperature in a neutral pH condition.
19. The composition as in any one of the preceding clauses, wherein the minicell stabilizes the active agent in a thermal variation.
20. The composition as in any one of the preceding clauses, wherein the active agent encapsulated by the minicell is at least 1.1 fold more resistant to thermal degradation than a free active agent not encapsulated by the minicell on day 7 after a heat treatment at 40° C.
21. The composition as in any one of the preceding clauses, wherein the active agent encapsulated by the minicell has less than about 60% thermal degradation on day 7 after a heat treatment at 40° C.
22. The composition as in any one of the preceding clauses, wherein the minicell protects the active agent from oxidative degradation by ultraviolet (UV) or visible light.
23. The composition as in any one of the preceding clauses, wherein the active agent encapsulated by the minicell is at least 1.1 fold more resistant to oxidative degradation than a free active agent not encapsulated by the minicell on day 7 under UV or visible light exposure.
24. The composition as in any one of the preceding clauses, wherein the active agent encapsulated by the minicell has less than about 35% oxidative degradation on day 7 under UV or visible light exposure.
25. The composition as in any one of the preceding clauses, wherein the minicell confers to the active agent an improved stability, an enhanced bioavailability and an extended shelf life.
26. The composition as in any one of the preceding clauses, wherein a release of the active agent encapsulated by the minicell is delayed when compared to a free active agent not encapsulated by the minicell.
27. The composition as in any one of the preceding clauses, wherein a release percentage (%) of the encapsulated active agent is less than about 50% in a first hour.
28. The composition as in any one of the preceding clauses, wherein a release percentage (%) of the encapsulated active agent is at least about 45% at 8 hours after the release.
29. The composition as in any one of the preceding clauses, wherein the encapsulated active agent has an extended release with less than about 50% of the active agent retained at 8 hours after the release.
30. The composition as in any one of the preceding clauses, wherein the minicell is coated by biopolymer.
31. The composition as in any one of the preceding clauses, wherein the biopolymer is a chitosan.
32. The composition as in any one of the preceding clauses, wherein a release of the active agent encapsulated by the biopolymer-coated minicell is further delayed when compared to the encapsulated active agent without the biopolymer coated.
33. The composition as in any one of the preceding clauses, wherein the active agent encapsulated by the biopolymer-coated minicell has a further extended release with at least about 10% of the active agent retained, when compared to the encapsulated active agent without the biopolymer coated, at 8 hours after the release.
34. The composition as in any one of the preceding clauses, wherein the active agent encapsulated by the minicell is capable of being delivered to a target in a controlled release manner.

### Bacterial Minicell Comprising an Essential Oil

1. A bacterial minicell comprising: an essential oil, wherein the minicell is loaded with the essential oil in a weight-to-weight ratio of about 5:1 to about 1:5, wherein about 50% w/w to about 150% w/w of the essential oil is encapsulated by the minicell, and wherein a release percentage (%) of the encapsulated essential oil is less than about 50% in a first hour.
2. The bacterial minicell as in any one of the preceding clauses, wherein a release percentage (%) of the encapsulated essential oil is at least about 45% at 8 hours after the release.
3. The bacterial minicell as in any one of the preceding clauses, wherein the encapsulated essential oil has an extended release with less than about 50% of the essential oil retained at 8 hours after the release.
4. The bacterial minicell as in any one of the preceding clauses, wherein the minicell is coated by biopolymer.
5. The bacterial minicell as in any one of the preceding clauses, wherein the biopolymer is a chitosan.
6. The bacterial minicell as in any one of the preceding clauses, wherein a release of the essential oil encapsulated by the biopolymer-coated minicell is further delayed when compared to the encapsulated essential oil without the biopolymer coated.
7. The bacterial minicell as in any one of the preceding clauses, wherein the essential oil encapsulated by the biopolymer-coated minicell has a further extended release with at least about 10% of the essential oil retained, when compared to the encapsulated essential oil without the biopolymer coated, at 8 hours after the release.
8. The bacterial minicell as in any one of the preceding clauses, wherein the essential oil encapsulated by the minicell is capable of being delivered to a target in a controlled release manner
9. The bacterial minicell as in any one of the preceding clauses, wherein the essential oil comprises geraniol, eugenol, genistein, thymol, pyrethrum or carvacrol.

### Method of Preparing a Minicell Encapsulating an Active Agent

1. A method of preparing an minicell encapsulating an active agent, said method comprising the steps of:
   a) producing and purifying minicells;
   b) providing an active agent;
   c) loading the minicells with the active agent for encapsulation; and
   d) recovering the minicells encapsulating the active agent.
2. The method as in any one of the preceding clauses, wherein in step a) the minicells are produced from a bacterial cell.
3. The method as in any one of the preceding clauses, wherein in step a) the purified minicells are provided as a suspension in water or other suitable liquid, or a concentrated paste.
4. The method as in any one of the preceding clauses, wherein the suspension comprises about 1 to 500 mg minicells per ml.
5. The method as in any one of the preceding clauses, wherein in step a) the purified minicells are provided as a dry powder.
6. The method as in any one of the preceding clauses, wherein in step b) the active agent provided as a suspension in an aqueous solvent.
7. The method as in any one of the preceding clauses, wherein in step c) the loaded minicells are suspended in a suspension of the active agent.
8. The method as in any one of the preceding clauses, wherein in step c) the reaction is carried out at atmospheric pressure at a temperature of about 1° C. to about 40° C.
9. The method as in any one of the preceding clauses, wherein in step c) the loading ratio between the minicells and the active agent is about 1:5 to about 5:1.
10. The method as in any one of the preceding clauses, further comprising the step of drying the minicells encapsulating the active agent.
11. The method as in any one of the preceding clauses, wherein the drying of the minicells encapsulating the active agent is by evaporating a solvent.
12. The method as in any one of the preceding clauses, wherein the active agent is a biologically active agent.
13. The method as in any one of the preceding clauses, wherein the biologically active agent is selected from a nucleic acid, a peptide, a protein, an essential oil, and combinations thereof.
14. The method as in any one of the preceding clauses, wherein the nucleic acid is selected from the group consisting of an antisense nucleic acid, a double-stranded RNA (dsRNA), a short-hairpin RNA (shRNA), a small-interfering RNA (siRNA), a microRNA (miRNA), a ribozyme, an aptamer, and combination thereof.
15. The method as in any one of the preceding clauses, wherein the terpene is geraniol, eugenol, genistein, carvacrol, thymol, pyrethrum or carvacrol.

### Method of Producing an Animal Feed for Enhancing Health of an Animal

1. A method of producing an animal feed for enhancing health of an animal, said method comprising: applying to an animal feed a composition as in any one of the preceding clauses.
2. The method as in any one of the preceding clauses, wherein the animal feed comprises at least one animal feed ingredient selected from the group consisting of a feed carbohydrate a feed protein, a fee vitamin and mixture thereof.
3. The method as in any one of the preceding clauses, wherein the animal health indicator is selected from the group consisting of: gut lesion formation, gut microbiota, weight change, feed conversion ratio, and life span.

### Method of Enhancing Health of an Animal

1. A method of enhancing health of an animal, said method comprising: administering to an animal in need thereof an effective amount of a composition as in any one of the preceding clauses.
2. The method as in any one of the preceding clauses, wherein the health of the animal administered with the composition is enhanced when compared to the health of the animal not administered with the composition.
3. The method as in any one of the preceding clauses, wherein the composition is administered with an animal feed.
4. The method as in any one of the preceding clauses, wherein the animal feed comprises at least one animal feed ingredient selected from the group consisting of a feed carbohydrate, a feed protein, a fee vitamin and mixture thereof.
5. The method as in any one of the preceding clauses, wherein the enhanced animal health is selected from the group consisting of: reduced or eliminated microbial infection, reduced or eliminated fungal infection, reduced or eliminated viral infection, reduced or eliminated oxidative stress, reduced or eliminated infection or death during transport, increased body weight, increased rate of weight gain, increased growth rate, reduced or eliminated birth mortality, increased or improved body score conditions, increased reproductive success, and increased gut health maturation.

### Method of Delivering an Active Agent to a Subject

1. A method of delivering an active agent to a subject, the method comprising: applying to the subject with a composition as in any one of the preceding clauses.
2. The method as in any one of the preceding clauses, wherein the subject is an animal.
3. The method as in any one of the preceding clauses, wherein the active agent is selected from a nucleic acid, a peptide, a protein, an essential oil, and combinations thereof.
4. The method as in any one of the preceding clauses, wherein the active agent has an antimicrobial, antibacterial, antifungal or antiviral activity.
5. The method of as in any one of the preceding clauses, wherein the active agent is a veterinary drug for animal vaccination or immunization.
6. The method as in any one of the preceding clauses, wherein the animal vaccination or immunization is against bacterial infections, gastric disorders, viral infections, cancer, parasite infections, non-infectious diseases, fertility and production control.

1. A composition comprising: a minicell and an active agent,
wherein the active agent is encapsulated by the minicell, and
wherein the minicell and the active agent are present in a weight-to-weight ratio of about 5:1 to about 1:5 in the composition.

2. The composition of claim 1, wherein the minicell and the active agent are present in a weight-to-weight ratio of about 1:1.

3. The composition of claim 1, wherein the minicell is derived from a bacterial cell.

4. The composition of claim 1, wherein the minicell is less than or equal to 1 μm in diameter.

5. The composition of claim 1, wherein the active agent is a biologically active agent.

6. The composition of claim 5, wherein the biologically active agent is selected from a nucleic acid, a peptide, a protein, an essential oil, and combinations thereof.

7. The composition of claim 6, wherein the nucleic acid is selected from the group consisting of an antisense nucleic acid, a double-stranded RNA (dsRNA), a short-hairpin RNA (shRNA), a small-interfering RNA (siRNA), a microRNA (miRNA), a ribozyme, an aptamer, and combination thereof.

8. The composition of claim 6, wherein the essential oil comprises geraniol, eugenol, genistein, carvacrol, thymol, pyrethrum or carvacrol.

9. The composition of claim 8, wherein the essential oil comprises geraniol.

10. The composition of claim 8, wherein the essential oil comprises eugenol.

11. The composition of claim 8, wherein the essential oil comprises genistein.

12. The composition of claim 8, wherein the essential oil comprises thymol.

13. The composition of claim 8, wherein the essential oil comprises pyrethrum.

14. The composition of claim 1, wherein at least about 0.1% of the active agent is encapsulated by the minicell.

15. The composition of claim 1, wherein the minicell stabilizes the active agent in an acidic condition, wherein the acidic condition is less than pH 7.

16. The composition of claim 15, wherein the minicell encapsulating the active agent is preserved from depletion flocculation when a pH is adjusted to an extremely acidic condition.

17. The composition of claim 16, wherein the acidic condition is as low as pH 1.

18. The composition of claim 15, wherein the minicell stabilizes the active agent at least 30 days at room temperature in a neutral pH condition.

19. The composition of claim 1, wherein the minicell stabilizes the active agent in a thermal variation.

20. The composition of claim 19, wherein the active agent encapsulated by the minicell is at least 1.1 fold more resistant to thermal degradation than a free active agent not encapsulated by the minicell on day 7 after a heat treatment at 40° C.

21. The composition of claim 20, wherein the active agent encapsulated by the minicell has less than about 60% thermal degradation on day 7 after a heat treatment at 40° C.

22. The composition of claim 1, wherein the minicell protects the active agent from oxidative degradation by ultraviolet (UV) or visible light.

23. The composition of claim 22, wherein the active agent encapsulated by the minicell is at least 1.1 fold more resistant to oxidative degradation than a free active agent not encapsulated by the minicell on day 7 under UV or visible light exposure.

24. The composition of claim 23, wherein the active agent encapsulated by the minicell has less than about 35% oxidative degradation on day 7 under UV or visible light exposure.

25. The composition of claim 1, wherein the minicell confers to the active agent an improved stability, an enhanced bioavailability and an extended shelf life.

26. The composition of claim 1, wherein a release of the active agent encapsulated by the minicell is delayed when compared to a free active agent not encapsulated by the minicell.

27. The composition of claim 26, wherein a release percentage (%) of the encapsulated active agent is less than about 50%in a first hour.

28. The composition of claim 26, wherein a release percentage (%) of the encapsulated active agent is at least about 45% at 8 hours after the release.

29. The composition of claim 28, wherein the encapsulated active agent has an extended release with less than about 50% of the active agent retained at 8 hours after the release.

30. The composition of claim 1, wherein the minicell is coated by biopolymer.

31. The composition of claim 30, wherein the biopolymer is a chitosan.

32. The composition of claim 30, wherein a release of the active agent encapsulated by the biopolymer-coated minicell is further delayed when compared to the encapsulated active agent without the biopolymer coated.

33. The composition of claim 32, wherein the active agent encapsulated by the biopolymer-coated minicell has a further extended release with at least about 10% of the active agent retained, when compared to the encapsulated active agent without the biopolymer coated, at 8 hours after the release.

34. The composition of claim 1, wherein the active agent encapsulated by the minicell is capable of being delivered to a target in a controlled release manner.

35.-57. (canceled)
